SEQUENCE LISTING (44 sequences)

SEQ ID NO: 1 - DNA, 20 bp, Homo sapiens
  source 1..20: mol_type = genomic DNA
aagatgggagggaattacgg

SEQ ID NO: 2 - DNA, 20 bp, Homo sapiens
  source 1..20: mol_type = genomic DNA
tggaggagttgccagttctt

SEQ ID NO: 3 - DNA, 20 bp, Homo sapiens
  source 1..20: mol_type = genomic DNA
ttccagaaggagctgaagga

SEQ ID NO: 4 - DNA, 20 bp, Homo sapiens
  source 1..20: mol_type = genomic DNA
cgctggccttttatttcgta

SEQ ID NO: 5 - DNA, 20 bp, Homo sapiens
  source 1..20: mol_type = genomic DNA
gaaggcaccaaactggatgt

SEQ ID NO: 6 - DNA, 20 bp, Homo sapiens
  source 1..20: mol_type = genomic DNA
gaagtaggccaggttggtca

SEQ ID NO: 7 - DNA, 22 bp, Homo sapiens
  source 1..22: mol_type = genomic DNA
ccagaaggatgatgtcgctcag

SEQ ID NO: 8 - DNA, 23 bp, Homo sapiens
  source 1..23: mol_type = genomic DNA
gagtaaggtccagaatgacacgg

SEQ ID NO: 9 - DNA, 23 bp, Homo sapiens
  source 1..23: mol_type = genomic DNA
gacagatagtccttcgctggaag

SEQ ID NO: 10 - DNA, 22 bp, Homo sapiens
  source 1..22: mol_type = genomic DNA
tctgaccacaggctgaagacca

SEQ ID NO: 11 - DNA, 22 bp, Homo sapiens
  source 1..22: mol_type = genomic DNA
gaggctatgtggcttcactgct

SEQ ID NO: 12 - DNA, 23 bp, Homo sapiens
  source 1..23: mol_type = genomic DNA
gacaatgtgagggcactccttag

SEQ ID NO: 13 - DNA, 24 bp, Homo sapiens
  source 1..24: mol_type = genomic DNA
ccctggaacaaagagatgggcgat

SEQ ID NO: 14 - DNA, 20 bp, Homo sapiens
  source 1..20: mol_type = genomic DNA
gattttggcgccccgttccc

SEQ ID NO: 15 - DNA, 20 bp, Homo sapiens
  source 1..20: mol_type = genomic DNA
cgaagtccccaaccacaagt

SEQ ID NO: 16 - DNA, 20 bp, Homo sapiens
  source 1..20: mol_type = genomic DNA
cgccctcgttcaccaaaaag

SEQ ID NO: 17 - DNA, 20 bp, Homo sapiens
  source 1..20: mol_type = genomic DNA
tccccttcatccctatgcca

SEQ ID NO: 18 - DNA, 20 bp, Homo sapiens
  source 1..20: mol_type = genomic DNA
cagagacccaccgaggtact

SEQ ID NO: 19 - DNA, 20 bp, Homo sapiens
  source 1..20: mol_type = genomic DNA
ccattagaaagcagttccgc

SEQ ID NO: 20 - DNA, 20 bp, Homo sapiens
  source 1..20: mol_type = genomic DNA
tgggtaggagatggagatgc

SEQ ID NO: 21 - DNA, 22 bp, Homo sapiens
  source 1..22: mol_type = genomic DNA
ttgcaggcttctccaactggac

SEQ ID NO: 22 - DNA, 22 bp, Homo sapiens
  source 1..22: mol_type = genomic DNA
cagaaccaggagcacagtgaag

SEQ ID NO: 23 - DNA, 22 bp, Homo sapiens
  source 1..22: mol_type = genomic DNA
ggatctccaacatggcagcctt

SEQ ID NO: 24 - DNA, 22 bp, Homo sapiens
  source 1..22: mol_type = genomic DNA
agacggctttctccctcttgct

SEQ ID NO: 25 - DNA, 23 bp, Homo sapiens
  source 1..23: mol_type = genomic DNA
ccgctttcatgtggaggaagaag

SEQ ID NO: 26 - DNA, 22 bp, Homo sapiens
  source 1..22: mol_type = genomic DNA
ctctgtgagcagtgccaaaagc

SEQ ID NO: 27 - DNA, 22 bp, Homo sapiens
  source 1..22: mol_type = genomic DNA
ttgccttgctgctctacctcca

SEQ ID NO: 28 - DNA, 22 bp, Homo sapiens
  source 1..22: mol_type = genomic DNA
gatggcagtagctgcgctgata

SEQ ID NO: 29 - DNA, 21 bp, Homo sapiens
  source 1..21: mol_type = genomic DNA
actacaaccgatccacctcac

SEQ ID NO: 30 - DNA, 21 bp, Homo sapiens
  source 1..21: mol_type = genomic DNA
actttgcctcccagatcacag

SEQ ID NO: 31 - DNA, 20 bp, Homo sapiens
  source 1..20: mol_type = genomic DNA
ttttccgaggatgagattgc

SEQ ID NO: 32 - DNA, 20 bp, Homo sapiens
  source 1..20: mol_type = genomic DNA
ctttccacatgctggctaca

SEQ ID NO: 33 - DNA, 20 bp, Homo sapiens
  source 1..20: mol_type = genomic DNA
tgaccagagcatccaaaaga

SEQ ID NO: 34 - DNA, 20 bp, Homo sapiens
  source 1..20: mol_type = genomic DNA
ctcttcgacctcgaaacagc

SEQ ID NO: 35 - DNA, 22 bp, Homo sapiens
  source 1..22: mol_type = genomic DNA
attgccgtgactgcctaccaga

SEQ ID NO: 36 - DNA, 22 bp, Homo sapiens
  source 1..22: mol_type = genomic DNA
ggaattgacagttgggtccagg

SEQ ID NO: 37 - DNA, 20 bp, Homo sapiens
  source 1..20: mol_type = genomic DNA
ggacttcgagcaagagatgg

SEQ ID NO: 38 - DNA, 20 bp, Homo sapiens
  source 1..20: mol_type = genomic DNA
agcactgtgttggcgtacag

SEQ ID NO: 39 - DNA, 22 bp, unidentified
  source 1..22: mol_type = genomic DNA
ggtctctgttcacgtcctcaag

SEQ ID NO: 40 - DNA, 22 bp, unidentified
  source 1..22: mol_type = genomic DNA
gaacaccagtgatggcacaggt

SEQ ID NO: 41 - DNA, 22 bp, unidentified
  source 1..22: mol_type = genomic DNA
ctgcctctgttttgcactacgc

SEQ ID NO: 42 - DNA, 22 bp, unidentified
  source 1..22: mol_type = genomic DNA
tcctcacagcagatcggcacat

SEQ ID NO: 43 - DNA, 20 bp, unidentified
  source 1..20: mol_type = genomic DNA
agccatgtacgtagccatcc

SEQ ID NO: 44 - DNA, 20 bp, unidentified
  source 1..20: mol_type = genomic DNA
ctctcagctgtggtggtgaa

(19) United States
(12) Patent Application Publication
(10) Pub. No.: US 2026/0076937 A1
(43) Pub. Date: Mar. 19, 2026

(54) COMPOSITION FOR PREVENTING OR TREATING AUTOIMMUNE DISEASES COMPRISING A BCAT1 INHIBITOR

(71) Applicant: Seoul National University R&DB Foundation, Seoul (KR)

(72) Inventors: Won-Woo Lee, Seoul (KR); Yeon Joon Kang, Seoul (KR)

(73) Assignee: Seoul National University R&DB Foundation, Seoul (KR)

(21) Appl. No.: 19/210,387

(22) Filed: May 16, 2025

(30) Foreign Application Priority Data
May 17, 2024 (KR) 10-2024-0064826
May 13, 2025 (KR) 10-2025-0062191

(51) Int. Cl. A61K 31/343; A61K 9/00; A61K 31/19; A61K 31/20; A61P 29/00

(52) U.S. Cl. CPC A61K 31/343; A61K 9/0053; A61K 31/19; A61K 31/20; A61P 29/00

(57) ABSTRACT

Disclosed herein are branched chain amino acid transaminase 1 (BCAT1) inhibitors and uses thereof. More specifically, disclosed are BCAT1 inhibitors that limit amino acid metabolism essential for CD4⁺ T-cell responses and their use in preventing, improving, or treating inflammatory disease or autoimmune disease. By identifying that BCAT1 acts as a major regulator in the inflammatory response of CD4⁺ T-cells, it is possible to regulate the inflammatory response by CD4⁺ T cells secreting interleukin-17 (IL-17), which is a major immune cell of autoimmune diseases, by utilizing a BCAT1 inhibitor.

### CROSS-REFERENCE TO RELATED APPLICATION

This application claims priority to Korean Patent Application No. 10-2024-0064826, filed on May 17, 2024, and Korean Patent application No. 10-2025-0062191, filed on May 13, 2025, the entire contents of which are incorporated herein by reference.

On the other hand, the present application was supported by the following national development project.

### [National Research and Development Project Supporting the Invention]

[Task Unique Number]1711180994
[Task Number]2022R1A2C3011243
[Ministry] Ministry of Science and ICT
[Name of Project Management (Professional) Institution] Korea Research Foundation
[Name of Research Project] Personal Basic Research (Ministry of Science and ICT)
Role and Mechanism of Solute Carrier (SLC) Membrane Transporter on Regulation of Inflammatory Response in Immune Cells
[Task Execution Organization Name] Seoul National University
[Study Period]2024 Mar. 1-2025 Feb. 28

### INCORPORATION BY REFERENCE STATEMENT OF THE MATERIAL CONTAINED IN THE “SEQUENCE LISTING XML” FILE

The Sequence Listing XML file submitted herewith is identified as follows:
Date of Creation: Oct. 1, 2025; Sequence File Name: 171081.00048_SeqList.xml; Size in bytes: 39,623 bytes.

### BACKGROUND

### 1. Field

Disclosed herein are branched chain amino acid transaminase 1 (BCAT1) inhibitors and uses thereof. More specifically, disclosed are BCAT1 inhibitors that limit amino acid metabolism essential for CD4⁺ T-cell responses and their use in preventing, improving, or treating inflammatory diseases, autoimmune diseases, and the like.

### 2. Description of the Related Art

T-cell activation is key to the adaptive immune response for host defense. This process is coupled with bioenergetic and biosynthetic needs to support proliferation, differentiation and cytokine production of T cells in response to antigen. Activated T cells undergo metabolically dynamic changes through a variety of mechanisms, including increases in glucose uptake and glucose metabolism, mitochondrial function, amino acid uptake, and lipid synthesis. Metabolic status critically regulates the functional plasticity of T-cells. According to accumulated research results, amino acids are essential nutrients for maintaining the high metabolic state of activated immune cells and supporting various immune cell functions. Expression of amino acid transporters, such as SLC7A5 (also referred to as L-type amino acid transporter 1, LAT1), is up-regulated to meet the specific amino acid needs of activated immune cells. Amino acids not only act as basic components for protein synthesis, but also directly function as nutrient signals that regulate signaling pathways or are converted to metabolic intermediates to participate in other intracellular metabolic pathways.

Branched-chain amino acids (BCAAs), including leucine, isoleucine, and valine, are essential for normal growth and development primarily due to their ability to promote protein synthesis through activation of the mTOR signaling pathway. In immune cells, mTOR plays an important role as a regulator of cellular metabolism and affects several functions, including proliferation, differentiation, and effector responses. Branched-chain aminotransferases (BCAT), which are the initial enzymes of BCAA degradation, reversibly transfer the amino group from BCAA to branched-chain α-keto acids (BKAs), which undergo oxidative decarboxylation by the branched-chain α-keto acid dehydrogenase complex (BCKDC) to form CoA derivatives and act as substrates for the TCA cycle to produce ATP. BCAT has two isozymes and is divided into mitochondrial BCAT (BCATm or BCAT2) and cytoplasmic BCAT (BCATc or BCAT1). BCAT2 is expressed in most tissues except the liver and is therefore considered a major enzyme in BCAA degradation, whereas the expression of BCAT1 is mainly confined to the nervous system. Since BCAT2 is widely expressed in several tissues and plays an important role in BCAA metabolism, studies on the function of BCAT2 in this process have been actively conducted. Recent studies have shown that BCAT1 is highly expressed in activated T cells and macrophages and is involved in regulating the inflammatory response. This suggests that BCAT1 may play an important role in regulating metabolic programming in activated immune cells. However, the effects of BCAT1-mediated metabolism of BCAAs, especially leucine, on T cell-mediated immune responses and their underlying molecular mechanisms remain unclear.

On the other hand, the proportion of patients with autoimmune diseases is gradually increasing due to changes in the industrialized and westernized living environment and the rapid transition to an aging society, which has emerged as a serious social problem mainly in developed countries. The global autoimmune disease treatment market is expected to reach about 200 trillion won by 2025, with an average annual growth rate of 4.2%. Accordingly, numerous domestic and foreign pharmaceutical companies have made various attempts to develop effective therapeutic agents. A representative method of alleviating symptoms of autoimmune diseases is to regulate the function of major immune cells that cause inflammation. Steroids, non-steroidal anti-inflammatory agents, immunosuppressants, and the like have been used for a long time to achieve this purpose, but serious side effects have occurred during long-term use, and the need to develop new therapeutic agents has emerged. Among the recently developed treatments for autoimmune diseases, infliximab and decemotinib are representative and are currently widely used. The infliximab is a neutralizing antibody that directly binds to tumor necrosis factor-α (TNF-α), a potent inflammation-inducing factor, and inhibits its function. However, since antibody-based drugs are protein preparations, there are difficulties in production, distribution, and storage processes, and the need for developing new therapeutic agents is further emphasized due to limitations such as side effects. The dechernotinib, developed as a chemical type of drug for treating autoimmune diseases, is a Janus kinase 3 (JAK3) inhibitor that prevents the JAK signaling system, which is important for the biological response of various cells and tissues, and thus various side effects have been reported in addition to the therapeutic effect. In particular, increased liver levels, increased blood fat levels, and abdominal pain and diarrhea are commonly reported, but are still used as therapeutic agents because they may be controlled with other drugs.

### SUMMARY

One object of the present invention is to provide a branched chain amino acid transaminase 1 (BCAT1) inhibitor and use thereof.

In order to achieve the above object,
the present invention provides, in one aspect, a composition for preventing, improving or treating an inflammatory disease; or an autoimmune disease, comprising a BCAT1 inhibitor.

In one aspect, the present invention includes an inhibitor of branched chain amino acid transaminase 1 (BCAT1) to alleviate the inflammatory response of CD4⁺ T-cells, and to reduce the induction and symptoms of autoimmune diseases without side effects. By identifying that BCAT1 acts as a major regulator in the inflammatory response of CD4⁺ T-cells, it is possible to regulate the inflammatory response by CD4⁺ T cells secreting interleukin-17 (IL-17), which is a major immune cell of autoimmune diseases, by utilizing a BCAT1 inhibitor.

In another aspect, the present invention may also be applied to various inflammatory diseases in which CD4⁺ T-cells are involved, since the effects of BCAT1 inhibitors on limiting the function of CD4⁺ T cells have been confirmed.

In still other aspect, the present invention has the advantage that administration of a BCAT1 inhibitor for a certain period of time (e.g., the first two weeks) when inflammation is induced may significantly reduce the severity of an inflammatory disease or an autoimmune disease without continuous administration thereafter.

In still other aspect, the present invention has the advantage of effectively regulating an immune response by targeting BCAT1 while being relatively less important in other cells and tissues, and thus specifically inhibiting an inflammatory response.

In still other aspect, the present invention may be utilized as an inhibitor against a target that does not show clinical symptoms even if a genetic mutation is present, and thus has high stability.

In still other aspect, the present invention is developed as a chemical-based drug, which has the advantage of solving problems in the production, distribution, and storage process of existing antibody-based therapeutic agents.

### BRIEF DESCRIPTION OF THE DRAWINGS

T-cell receptor (TCR) stimulation induces expression of BCAT1 and solute carrier family 7 member 5 (SLC7A5) in human CD4⁺ T cells. FIGS. 1A and 1B are each the result of analysis of mRNA and protein expression of BCAT1 and BCAT2 in human CD4⁺ T cells of healthy control (HC) through RT-qPCR (A; n=5) and immunoblotting (B; n=3) at 24 hours with or without TCR stimulation. FIG. 1C is the result of examining the expression of 42 amino acid transporters at designated time points in TCR-stimulated human CD4+ memory T cells by analyzing Public RNA-seq data (GEO number: GSE140244). This relates to a heatmap analysis (left) and fold change (right) of amino acid transporter expression at designated times after TCR stimulation. FIG. 1D shows the results of validating the expression of the major BCAA transporters in human CD4+ T cells of HC (n=5) via RT-qPCR at 24 hours post-stimulation. FIG. 1E is the result of analyzing protein expression of SLC7A5 in human CD4+ T cells derived from HC at 24 hours depending on TCR stimulation (n=3). FIG. 1F relates to ³H-leucine uptake by TCR-stimulated human CD4+ T cells in the presence of 50 mM BCH and 10 μM JPH203 (n=5). The graph shows the mean±standard error (SEM). A significance was indicated as *p<0.05, **p<0.01, and ***p<0.001, according to Mann-Whitney U test (a, d, f), two-tailed non-pair t test (b, e), or one-way ANOVA (f) with Tukey test.

mRNA expression of SLC7A5 is preferentially induced by TCR-stimulated CD4 T cells. The mRNA expression of SLC7A5 in FIG. 2A was analyzed by RT-qPCR at 24 hours after TCR stimulation in human CD4+ T cells and CD8+ T cells of HC (n=8-11). FIG. 2B relates to ³H-methionine uptake by TCR-stimulated human CD4⁺ T cells with BCH or JPH203 (n=5). The graph shows the mean±SEM. The significance was indicated as ***=p<0.001 and ****=p<0.001, according to Mann-Whitney U test (a) or Turkey's one-way ANOVA (b).

FIG. 3 relates to the effect of BCAT1 inhibition (Bi2) on pharmacological response in HepG2 cells. Cell levels of α-ketoisocarproic acid (α-KIC), a metabolite of BCAT, were assessed in HepG2 cells under leucine supplementation or Bi2 treatment conditions. Quantitative representations of α-KIC levels are shown (n=3). The graph shows the mean±SEM. The significance was indicated as **=p<0.01 and ****=p<0.0001, according to two-tailed non-paired t-test.

SLC7A5-mediated leucine influx regulates the Th17 response. FIG. 4A shows mRNA expression of SLC7A5, BCAT1 and BCAT2 in human naive and memory CD4⁺ T cells (n=5) at 24 h post-stimulation by RT-qPCR. FIG. 4B is stimulation of CFSE-labeled naive and memory CD4+ T cells with anti-CD3/CD28 coated microbeads for 4 days with or without BCAA, Bi2 (10 μM), or JPH203 (10 μM) (n=5). The proportion of proliferating cells was determined by CFSE dilution analysis. FIGS. 4C and 4D relate to each the amount of cytokines (n=5) in culture supernatants of CD4⁺ naive T cells (c) and CD4⁺ memory T cells (d) stimulated with TCRs stimulated with anti-CD3/CD28 coated microbeads for 3 days under the specified conditions. FIG. 4E relates to the amounts of IL-17A (left) and IFN-γ (right) (n=7-8) in culture supernatants of TCR-stimulated CD4⁺ memory T cells supplemented with leucine and Bi2. Freshly isolated human CD4⁺ memory T cells were activated and infected with GFP lentivirus with BCAT1 shRNA for 24 hours. GFP-expressing ShRNA⁺ cells were sorted and cultured for 3 more days under TCR stimulation. FIG. 4F relates to BCAT1 expression (n=5) (f) in sorted shRNA⁺ cells. The mRNA (left) and protein (right) levels of IL-17 and IFN-γ in the culture supernatant were analyzed by RT-qPCR (n=5) and ELISA (n=5) (g). The graph shows the mean±SEM. The significance was indicated as *p<0.05 and **p<0.01, according to Mann-Whitney U test.

BCAAs are involved in the regulation of effector function of CD4⁺ memory T cells. In FIGS. 5A to 5C, CFSE-labeled or -unlabeled CD4⁺ memory T cells were stimulated with anti-CD3/CD28 coated microbeads in leucine, isoleucine or valine-depleted media for 5 days (n=3-5). Cell viability (a) was analyzed by 7-AAD staining using a flow cytometer (n=3). The proportion (b) of proliferating cells was determined by CFSE dilution analysis (n=3). The amounts (c) of IL-17A and IFN-γ in culture supernatants of TCR-stimulated CD4⁺ memory T cells under the specified conditions were determined by ELISA (n=5). FIG. 5D shows CD4⁺ memory T cells were stimulated with anti-CD3/CD28 coated microbeads for 18 hours in BCAA-depleted media. Cells were supplemented with leucine, isoleucine or valine for 30 minutes before obtaing. Cell lysates were prepared and immunoblotted with phosphor p70-S6K and total p70-S 6K (n=3 independent experiments). The graph shows the mean±SEM. The significance was indicated as *=p<0.05 and **=p<0.01, according to the two-tailed non-pair t test (b) or the Mann-Whitney U test (c).

The BCAT1-mediated leucine metabolite, 3-hydroxy-3-methylbutyric acid (HMB) is involved in the regulation of the Th17 response. FIG. 6A examines the expression of BCAA degrading enzymes at specified times in TCR-stimulated human CD4⁺ memory T cells by analyzing Public RNA-seq data (GSE140244). This is a heatmap analysis results showing the time course of changes in expression of key BCAA degrading enzymes. FIG. 6B validates the expression of HPD, HPDL and BCKDK in human CD4⁺ T cells by RT-qPCR at 24 hours post-stimulation (n=4). FIG. 6C is a schematic of cytoplasmic leucine metabolism. In FIG. 6D CD4⁺ memory T cells were pretreated with Bi2 (10 μM), statin (10 μM) or HMB (0.4 mM) for 1 hour and then stimulated with anti-CD3/CD28 coated microbeads for 3 days. Cholesterol accumulation in cell lysates was measured (n=3). In FIG. 6E, the amount of IL-17A in the culture supernatant of TCR-stimulated CD4⁺ memory T cells was measured on day 3 by ELISA (n=4). TCR-stimulated CD4⁺ memory T cells in FIG. 6F were cultured with Bi2 and the indicated concentrations of acetate for 3 days. The amount of IL-17A was determined by ELISA (n=5). In FIG. 6G, the amount of IL-17A in the culture supernatant of CD4⁺ memory T cells supplemented with HMB was determined by ELISA (n=5). Human CD4⁺ memory T cells freshly isolated in FIGS. 6H and 6I were activated and infected with GFP lentivirus with HPD or HPDL shRNA for 24 hours. GFP-expressing ShRNA⁺ cells were sorted and cultured for 3 more days with TCR stimulation. FIG. 6H relates to expression of the indicated genes (n=3) in sorted shRNA⁺ cells. FIG. 6I shows the mRNA (left) and protein (right) levels of IL-17 and IFN-γ in culture supernatants analyzed by RT-qPCR (n=3) and ELISA (n=3). The graph shows the mean±SEM. The significance was indicated as *p<0.05, **p<0.01, ***p<0.001, ****p<0.0001, according to the Mann-Whitney U test (b, e), the one-way ANOVA and Kruskal-Wallis test (d, f, g), or the two-tailed non-pair t test (h, i), respectively.

BCAT1-mediated leucine metabolites have different effects on IFN-γ production in CD4⁺ memory T cells compared to IL-17A production. In FIG. 7A, TCR-stimulated CD4⁺ memory T cells were cultured with Bi2 (10 μM) and the indicated concentrations of acetate for 3 days. The amount of IFN-γ was determined by ELISA (n=5). FIG. 7B shows that the amount of IFN-γ in the culture supernatant of CD4⁺ memory T cells with HMB supplementation was determined by ELISA (n=5). The graph shows the mean SEM. The significance was indicated as *=p<0.05 and ***=p<0.001, according to one-way ANOVA using the Kruskal Wallis test

Multiple scRNA-seq analysis for three different groups of human CD4⁺ memory T cells in FIG. 8 was performed using Seurat in R software (version 4.3.0): TCR stimulation without TCR (TCR−), TCR stimulation without Bi2 for 72 hours (TCR+Bi2−), TCR stimulation with Bi2 (10 μM) for 72 hours (TCR+Bi2+). In FIG. 8A, individual cells (28.651 cells) were color-coded based on clusters (n=31) in a t-distributed stochastic neighbor (t-SNE) plot generated by unsupervised Seurat clustering. In FIG. 8B, the major CD4⁺ memory T cell subsets were identified through formal cell type marker expression. FIG. 8C relates to t-SNE plots isolated based on major CD4⁺ memory T cell subsets (top, TCR−, n=10,109 cells; TCR+Bi2−, n=9506 cells; TCR+Bi2+, n=9036 cells). The dashed region highlights Th17 cluster changes after stimulating TCR with Bi2 treatment. This is a pie chart showing relative Th subset abundance in various conditions. Activated T cells were annotated with mean expression of the activation markers listed in FIG. 7A. FIG. 8D relates to pathway enrichment analysis of differentially expressed genes (DEGs) in activated Th17 cells between TCR+Bi2− and TCR+Bi2+ groups. Representative genes of each pathway were indicated. FIG. 8E relates to Volcano plots for DEG of activated Th17 cells between TCR+Bi2− and TCR+Bi2+ groups. Volcano plots were generated using the EnhancedVolcano package (version 1.16.0). FIG. 8F shows HIF1A expression in activated Th17 cells projected onto the t-SNE plot (top) and plotted as a feature plot (top). Violin plot showing distribution of HIF1A expression levels, dots represent individual cells (bottom). The Gene Set Enrichment Analysis (GSEA) in FIG. 8G revealed 15 pathways enriched in 23,457 DEGs in the activated Th17 cluster with FDR<0.25. Red bars represent sets of genes with nominal p values<0.1. In FIG. 8H, GSEA was used to investigate a significantly enriched pathway. This indicates GSEA abundance plot (left) and heatmap (right) of DEG down-regulated in PI3K-AKT-mTOR signaling pathway in Bi2-treated cells. All transcripts in the annotated genes were uploaded to a locally installed GSEA tool and compared to the Hallmark gene set.

BCAT1 blockade is associated with a distinct transcript profile in activated Th17 T cells. FIG. 9A is a dot-plot showing the expanded mean gene expression of key marker genes of CD4⁺ memory T cells in each cluster (FIG. 8A). The color indicates expression and the size of the circle indicates the proportion of cells expressed. FIGS. 9B and 9C relate to each expression pattern of leucine metabolism-related genes in different subsets of CD4⁺ T cells. The dot plot shows the expanded mean gene expression of each gene (b) and HIF1α (c) within the CD4⁺ memory T cell population classified as Th1, Th17 or Treg clusters (see FIG. 8A). The color indicates the expression level, and the size of the circle indicates the proportion of cells expressing the corresponding gene. In FIG. 9D, the expression of the major marker gene of activated Th17 cells was projected onto a t-SNE plot labeled FeaturePlots (top). Violin plots show the distribution of expression levels of genes and dots represent individual cells (below).

HMB regulates HIF-1α expression in human CD4⁺ T cells. FIG. 10A is the result of analyzing protein expression of HIF-1α in TCR-stimulated CD4⁺ memory T cells treated with or without Bi2 (n=5). As a control for HIF-1α, PANC-1 cells were cultured in 2% or 21% O₂ for 8 hours. This indicates representative immunoblots (left). Band intensities of immunoblots were quantified densitometrically except for PANC-1 data. In FIG. 10B, a β-Actin was used as a normalization control (right). Human CD4⁺ memory T cells freshly isolated were activated and infected with GFP lentivirus with BCAT1, HPD or HPDL shRNA for 24 hours. The GFP-expressing shRNA⁺ cells were sorted and cultured for another 3 days under TCR stimulation. The mRNA expression of HIF-1α was analyzed by RT-qPCR (n=3-5). FIG. 10C shows that HIF-1α expression in human CD4⁺ memory T cells was measured 24 hours after TCR stimulation with Bi2 (n=5). Cells were supplemented with HMB (0.4 mM) for the specified time before obtaining. FIG. 10D is a quantitative PCR analysis (n=6) of HIF-1α target gene expression after 72 hours of TCR stimulation in human CD4⁺ memory T cells derived from HC. HMB (0.4 mM) was used to pretreat cells for 1 hour prior to TCR stimulation. In FIG. 10E, IL-17A and IFN-γ production was quantified 72 hours after TCR stimulation with or without Bi2 (10 μM), VH298 (100 nM) or HMB (0.4 mM) (n=5). In FIG. 10F, the mRNA expression of HIF-1α was measured 4 hours after TCR stimulation. Cells were supplemented (n=5) with HMB (0.4 mM) for the specified time before obtaining. FIG. 10G shows that CD4⁺ memory T cells were stimulated with anti-CD3/CD28 coated microbeads for 1 hour in the absence or presence of Bi2. Cell lysates were prepared at the specified times and immunoblotted with phosphorylated-p70-S6K and total p70-S6 K (n=3 independent experiments). FIG. 10H shows CD4⁺ memory T cells stimulated with anti-CD3/CD28 antibody for 1 hour. Cells were supplemented with HMB (0.4 mM) for the specified time before obtaining. Cell lysates were prepared at 1 h post-stimulation to perform immunoblots for phosphorylation-p70-S6K and total p70-S6 K (n=5 independent experiments). The graph shows the band intensities quantified by densitometry. The graph shows the mean±SEM. The significance was indicated as *p<0.05 and **p<0.01 according to the Mann-Whitney U test [a, b (left), c-f, h] or the two-tailed unpaired t test [b (right)].

BCAT1 regulates HIF-1α through mTORC1 activation in human CD4⁺ T cells. FIG. 11A relates to ubiquitin-proteasome degradation of HIF1a and inhibitors for each step. In FIG. 11B, CD4+ memory T cells were pretreated with Bi2 (10 μM), CoCl2 (100 μM), VH298 (100 μM) or MG132 (5 μM) for 1 hour and stimulated with anti-CD3/CD28 coated microbeads for 8 hours. Cell lysates were immunoblotted against HIF-1α (n=3 independent experiments). In FIG. 11C, CD4+ memory T cells were stimulated for 24 hours and then treated with CHX (0.2 μg/ml) for the specified time before being obtained. Cell lysates were immunoblotted against HIF-1α (n=3 independent experiments). In FIG. 11D, CD4⁺ memory T cells were stimulated for a specified time with or without Bi2 (10 μM). Cell lysates were immunoblotted (n=4 independent experiments) for phosphor-p70-S6K and total p70-S6 K. The graph shows the band intensities quantified by densitometry. In FIG. 11E, CD4+ naive T cells were pretreated with Bi2 (10 μM) for 1 hour followed by stimulation with anti-CD3/CD28 antibody for 1 hour. Cell lysates were immunoblotted (n=5 independent experiments) for phosphor-p70-S6K and total p70-S6 K. In FIG. 11F, CD4⁺ naive T cells were stimulated for 24 hours in the presence of a specified concentration of Bi2. Cell lysates were immunoblotted against HIF-1α (n=3 independent experiments). In FIG. 11G, CD4⁺ naive T cells were stimulated with anti-CD3/CD28 coated microbeads with Bi2 (10 μM) for 4 days. Cell viability was analyzed by 7-AAD staining using a flow cytometer (n=3). In FIG. 11H, human CD4⁺ naive T cells were isolated and treated with Bi2 (10 μM) for 1 hour prior to TCR stimulation for differentiation under various cytokine conditions (Th0, Th1, Th17 or iTreg). The mRNA levels of genes involved in leucine metabolism were quantified using RT-qPCR (n=3). In FIGS. 11I to 11L, CD4+ naive T cells were pretreated with Bi2 (10 μM) for 1 hour and differentiated under Th17 or Th1 polarization conditions for 7 days. The mRNA expression of the signature genes of Th17 (i-j) or Th1 (k-l) and cytokines was measured by RT-qPCR and ELISA, respectively (n=5-7). The graph shows the mean±SEM. The significance was indicated as *=p<0.05, **=p<0.01, ***=p<0.001, according to Mann Whitney U test.

BCAT1 inhibition alleviates the severity of experimental autoimmune encephalomyelitis (EAE). EAE was induced by MOG₃₅₋₅₅ in a CFA emulsion with pertussis toxin (PTX). Bi2 (10 mg/kg) was administered intrapentoneally to MOG-immunized mice 4 hours prior to immunization, and treatment was repeated three times a week for 14 days. FIG. 12A relates to clinical scores of EAE mice. FIG. 12B relates to histological analysis of spinal cord tissue stained with Luxol fast blue or Hematoxylin & Eosin on day 16. The areas of demyelination (left) and inflammatory cell infiltration (right) are indicated by black dotted lines. FIG. 12C quantifies immune cell infiltration in the spinal cord (SCMC: spinal cord monocytes) in four groups. FIG. 12D relates to the proportion of CD3+CD4+ T cells in total CD45+ T cells (n=6 per group) in untreated or Bi2 treated EAE mice. FIGS. 12E and 12F are each flow cytometry result of CD3+CD4+ T cells producing IL-17A and IFN-γ in SCMC and in inguinal lymph nodes (iLN) of EAE mice (n=5-8). In FIG. 12G, cell lysates were prepared in SCMC of EAE mice treated with DMSO or Bi2 and immunoblotted for HIF-1α (n=5 per group). FIG. 12H relates to representative histograms of intracellular HIF-1α in total CD45+ SCMC (left) and CD3+CD4+ T cells (right) of EAE mice (n=5 per group). The graph shows the mean±SEM. The significance was indicated as *p<0.05, **p<0.01, ***p<0.001 and ****p<0.0001, according to two-way ANOVA (a) or Mann-Whitney U test (c-h).

Blocking BCAT1 with Bi2 attenuates Th17 differentiation in mice. In FIG. 13A mRNA expression of slc7a5 and bcat1 in mouse inguinal LN (iLN) cells was analyzed by RT-qPCR at 24 hours after TCR stimulation with anti-CD3/CD28 antibody (n=5). In FIG. 13B protein expression of BCAT1 in mouse inguinal LN cells was analyzed at 24 hours after stimulation of TCR with anti-CD3/CD28 antibody (n=5). In FIGS. 13C to 13D, mouse CD4⁺ naive T cells derived from iLN were differentiated under Th7 or Th1 polarization conditions for 5 days in the presence of Bi2 (10 μM). The amounts of IFN-γ (c) and IL-17A (d) in the culture supernatants of Th1 and Th17 cells (n=5) are shown. In FIG. 13E, the cells of mouse iLN were stimulated with anti CD3/CD28 antibody in the presence of Bi2 (10 μM) for 24 hours. Cell lysates were immunoblotted for HIF-1α (n=3 independent experiments). Cells treated with COCl₂ served as a positive control for HIF-1α. The graph shows the band intensities quantified by densitometry. FIG. 13F is a representative histogram plot of intracellular HIF-1α in cells of mouse iLN. Cells were stimulated with anti CD3/CD28 antibody in the presence of Bi2 (10 μM) for 24 hours. The graph shows the mean±SEM. The significance was indicated as *=p<0.05, **=p<0.01, ***=p<0.001, according to Mann-Whitney U test (a, c, d, f) or the two-tailed non-pair t test (b, e)).

BCAT1 inhibition improves EAE induction. Experimental autoimmune encephalomyelitis (EAE) was induced by MOG35-55 in a CFA emulsion with PTX. The Bi2 (10 mg/kg) was administered intraperitoneally to MOG immunized mice 4 hours prior to immunization and treated repeatedly three times a week until day 14. FIG. 14A relates to the body weight of mice (n=5 per group). FIG. 14B relates to a gating strategy of flow cytometry to identify different immune cell subsets in spinal cord mononuclear cells (SCMCs). In FIG. 14C, the absolute number and frequency of each subset in SCMC were analyzed by flow cytometry (n=6 per group). FIG. 14D is a representative histogram plot of intracellular HIF-11α in CD11b+F4/80+ macrophages of the CD45+ population in SCMC of EAE mice (n=5 per group). The graph shows the mean±SEM. The significance was indicated as *=p<0.05 and **=p<0.01, according to Mann-Whitney U test.

LβhL, a leucine analogue, attenuates IL-17A production. FIG. 15A relates to ³H-leucine uptake (n=3) by TCR-stimulated CD4+ T cells in the presence of L-leucine, D-leucine or LβhL (400 mg/L each). In FIGS. 15B and 15C, the CFSE-labeled CD4+ naive (b) and memory (c) T cells were stimulated with anti-CD3/CD28 coated microbeads with or without L-leucine, LβhL (400 mg/L) or Bi2 (10 μM) (n=5). The proportion of proliferating cells was determined by CFSE dilution assay. FIG. 15D relates to the amount of cytokines (n=5) in the culture supernatant of CD4⁺ naive T cells (left) stimulated with TCR and CD4⁺ memory T cells (right) stimulated with anti-CD3/CD28 coated microbeads at the indicated conditions for 3 days. Cells were treated with Bi2 and LβhL for 1 hour prior to TCR stimulation. FIG. 15E relates to clinical scores of EAE mice. Bi2 (10 mg/kg) or LβhL (1 g/kg) was administered intraperitoneally to MOG immunized mice 4 hours prior to immunization, and treatment was repeated 3 times a week for 14 days. FIG. 15F shows flow cytometry (n=5 per group) results of CD3⁺ CD4⁺ T cells producing IL-17A and IFN-γ in iLN of EAE mice. FIG. 15G relates to SCMC quantification (n=6-7 per group) in three groups of EAE mice. FIG. 15H shows flow cytometry (n=5 per group) results of CD3+CD4+ T cells producing IL-17A and IFN-γ in SCMC of EAE mice. FIG. 15I relates to representative histogram plots (n=5 per group) for intracellular HIF-1α of CD3⁺CD4⁺ T cells in CD45⁺ SCMC of EAE mouse. The graph shows the mean±SEM. The significance was indicated as **p<0.01, ***p<0.001, and ****p<0.0001, according to one-way ANOVA using Tukey test (a). Mann-Whitney U test (b, c-f, g-i) or two-way ANOVA (e).

LβhL, a leucine analogue, improves EAE induction to a similar extent as Bi2-treated mice. FIG. 16 relates to histological analysis of spinal cord tissue stained with Luxol fast blue or Hematoxylin & Eosin. Demyelinating areas (top) and inflammatory cell infiltration (bottom) are indicated by dashed black lines.

In FIG. 17 freshly isolated CD4⁺ memory T cells from healthy control PBMCs were pretreated with indicated concentrations of gabapentin for 30 minutes and then cultured with aCD3/28 dynabeads (bead to cell ratio 1:10) for 72 hours. The graph shows the mean±SEM. The significance was indicated as *=p<0.05 according to one-way ANOVA. CD4⁺ memory T cells isolated from HCPBMCs, 5*10⁵ cells/well (% well U-bot) gabapentin 30 min pretreatment, aCD3/28 Dynabead 1:10 72 h, **Gabapentin: BCAT1 inhibitor.

Bi2 alleviates arthritis induction. FIG. 18A relates to an SKG mouse model in which arthritis was induced by curdlan. Bi2 was administered by intraperitoneal injection (i.p.) twice a week for 3 weeks after curdlan intraperitoneally (i.p) administration. FIG. 18B relates to clinical scores of arthritis (n=4 per group). FIG. 18C shows mRNA expression of arthritis-related genes in synovial cells analyzed by RT-qPCR (n=5). FIG. 18D shows flow cytometry measurements of CD3+CD4+ T cells producing IL-17A and IFN-γ in inguinal lymph nodes (iLNs) of SKG mice (n=5 per group). The graph shows the mean SEM. The significance was indicated as *=p<0.05 and **=p<0.01, according to two-way ANOVA (a) or Mann-Whitney U test (b-d) with significant interaction between time and Bi2.

### DETAILED DESCRIPTION

Hereinafter, the present invention will be described in detail.

According to the present invention, 3-hydroxy-3-methylbutyric acid (HMB), a branched chain amino acid transaminase 1 (BCAT1)-mediated leucine metabolite, has been shown to increase the Th17 response mainly through regulation of the mTORC1-HIF1α pathway, which is the main signaling pathway for IL-17 production. In addition, BCAT1 inhibition using BCATc inhibitor 2 (Bi2) reduced HIF1α expression and IL-17 production in T cells of the spinal cord, showing the effect of alleviating the severity of disease in an experimental autoimmune encephalomyelitis (EAE) mouse model. These results suggest that SLC7A5-mediated amino acid influx and BCAT1-mediated leucine degradation play an important role in regulating CD4⁺ T cell responses, particularly IL-17 production, through HIF1α regulation. Furthermore, these mechanisms may be associated with a variety of inflammatory conditions.

The term “administration” as used herein means to provide a composition according to the invention to a subject of administration in any suitable manner, and includes administration, absorption and ingestion, and the like. In this case, the subject of application refers to all animals, such as humans, monkeys, dogs, goats, pigs, and mice, to which the composition may be applied.

The term “prevention” as used herein refers to any action that inhibits or delays a condition or disease by administration of one embodiment of the invention. The term “treatment” refers to any act of ameliorating or beneficially altering the symptoms of an individual suspected of and developing a condition or disease by the administration of one embodiment of the present invention. The term “improvement” refers to any action in which a condition or disease is ameliorated or beneficially altered by administration of one embodiment of the invention.

The present invention relates in one aspect to a composition for preventing, improving or treating an inflammatory disease; or an autoimmune disease, comprising a BCAT1 inhibitor.

The present disclosure relates in another aspect to a method for preventing, improving or treating an inflammatory disease: or an autoimmune disease, comprising administering to a subject a composition comprising an effective amount of a BCAT1 inhibitor.

In one exemplary embodiment, the inflammatory disease or autoimmune disease may include, but is not limited to, autoimmune encephalomyelitis, multiple sclerosis, rheumatoid arthritis, systemic lupus erythematosus, type 1 diabetes, inflammatory bowel disease, Crohn's disease, ulcerative colitis, asthma, and atopic dermatitis, and the like, and may include various diseases associated with the inflammatory response of CD4⁺ T-cells.

In one exemplary embodiment, the BCAT1 inhibitor may include, but is not limited to, compounds such as BCATc inhibitor 2 represented by Formula 1, gabapentin represented by Formula 2, and/or ERG240 represented by Formula 3, and may include various forms of inhibitors capable of inhibiting BCAT1 activity. For example, the BCAT1 inhibitor may include not only a compound (e.g., a small molecule inhibitor), but also an RNA-based therapeutic agent capable of regulating BCAT1 expression (e.g, siRNA, shRNA, antisense oligonucleotide (ASO), etc.) and an antibody therapeutic agent targeting a BCAT1 protein (e.g; monoclonal antibody or nanobody, etc.). The inhibitors may be used alone or in combination with other immunomodulators or anti-inflammatory agents.

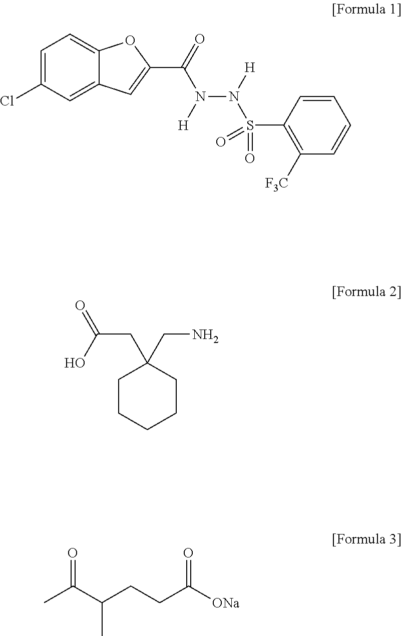

In one exemplary embodiment, the BCAT1 inhibitor may be administered at a dosage of 0.1 to 100 mg/kg/day. When the dosage of the BCAT1 inhibitor is less than 0.1 mg/kg/day, the effect of preventing, improving, or treating an inflammatory disease; or an autoimmune disease may be insignificant, and when the dosage is more than 100 mg/kg/day, the cell toxicity may be exhibited, or the efficiency of preventing, improving, or treating the inflammatory disease: or the autoimmune disease, may be reduced. More specifically, the daily dosage of the BCAT1 inhibitor may be, but is not limited to, 0.1 mg/kg/day or more, 0.15 mg/kg/day or more, 0.2 mg/kg/day or more, 0.25 mg/kg/day or more, 0.3 mg/kg/day or more, 0.35 mg/kg/day or more, 0.4 mg/kg/day or more, 0.45 mg/kg/day or more, 0.5 mg/kg/day or more, 0.55 mg/kg/day or more, 0.6 mg/kg/day or more, 0.65 mg/kg/day or more, 0.7 mg/kg/day or more, 0.75 mg/kg/day or more, 0.8 mg/kg/day or more, 0.85 mg/kg/day or more, 0.9 mg/kg/day or more, 0.95 mg/kg/day or more, 1.0 mg/kg/day or more, 2.0 mg/kg/day or more, 3.0 mg/kg/day or more, 4.0 mg/kg/day or more, [symbol] 5.0 mg/kg/day or more, or 100 mg/kg/day or less, 95 mg/kg/day or less, 90 mg/kg/day or less, 85 mg/kg/day or less, 80 mg/kg/day or less, 75 mg/kg/day or less, 70 mg/kg/day or less, 65 mg/kg/day or less, 60 mg/kg/day or less, 55 mg/kg/day or less, 50 mg/kg/day or less, 45 mg/kg/day or less, 40 mg/kg/day or less, 35 mg/kg/day or less, 30 mg/kg/day or less, 25 mg/kg/day or less, 20 mg/kg/day or less, 15 mg/kg/day or less, or 10 mg/kg/day or less. The administration may be divided into one to several times per day. For example, it may be administered 2 to 24 times per day, 1 to 2 times per 3 days, 1 to 6 times per week, 1 to 10 times per 2 weeks, 1 to 15 times per 3 weeks, 1 to 3 times per 4 weeks, or 1 to 12 times per year, but is not limited thereto.

In one exemplary embodiment, the composition may be a health functional food composition for preventing or improving autoimmune diseases.

In one exemplary embodiment, the composition may be a pharmaceutical composition for preventing or treating an autoimmune disease.

In one exemplary embodiment, when the composition is used as an additive of the health functional food, it may be added as it is or used together with other foods or food ingredients, and may be appropriately used according to a conventional method. The mixed amount of the active ingredient may be suitably determined according to each purpose of use such as prevention, health, or treatment. Formulations of food are possible in the form of a powder, granule, pill, tablet, or capsule as well as in the form of a general food or beverage.

In one exemplary embodiment, the type of the health functional food is not particularly limited, and examples of the food to which the composition may be added include meat, confectionery, noodles, gums, dairy products including ice creams, various soups, beverages, teas, drinks, alcoholic beverages, vitamin combinations, and the like, and may include all foods in a conventional sense.

In one exemplary embodiment, the beverage in the health functional food may contain various flavoring agents or natural carbohydrates and the like as an additional component as in a conventional beverage. The natural carbohydrates described above may be monosaccharides such as glucose, fructose, disaccharides such as maltose, sucrose and polysaccharides such as dextrin, cyclodextrin, sugar alcohols such as xylitol, sorbitol, erythritol and the like. As the sweetener, a natural sweetener such as thaumatin or stevia extract, or a synthetic sweetener such as saccharin or aspartame, or the like may be used. The proportion of the natural carbohydrates may be, but is not limited to, about 0.01 to 0.04 g, preferably about 0.02 to 0.03 g per 100 mL of the beverage according to the invention.

In one exemplary embodiment, in addition to the above, the health functional food according to the present invention may contain various nutrients, vitamins, electrolytes, flavoring agents, coloring agents, pectic acid and salts thereof, alginic acid and salts of thereof, organic acids, protective colloidal thickeners, pH adjusters, stabilizers, preservatives, glycerin, alcohols, and carbonating agents used in carbonated beverages. In addition, the food according to the invention may furthermore contain pulp for the production of natural fruit juices, fruit juice beverages and vegetable beverages. These components may be used independently or in admixture. The proportion of such additives is not limited but is generally selected in the range of 0.01 to 0.1 parts by weight relative to 100 parts by weight of the health functional food according to the present invention.

In one exemplary embodiment, the pharmaceutical composition may be provided in any formulation suitable for local administration. For example, it may be administered orally, transdermally, intravenously, intramuscularly, or subcutaneously. As an example, the pharmaceutical composition may be, but is not limited to, an injection, a solution for external use on the skin, a suspension, an emulsion, a gel, a patch, or a spray. The formulations may be readily prepared according to conventional methods in the art, and surfactants, excipients, hydrating agents, emulsification promoters, suspending agents, salts or buffers for osmoregulation, coloring agents, spices, stabilizers, preservatives, preserving agents or other compatible auxiliaries may be used as appropriate.

In one exemplary embodiment, the active ingredient of the pharmaceutical composition will vary depending on the age, sex, weight, pathology and severity of the subject, the route of administration or the judgment of the prescriber. The determination of the appropriate dose of use based on these factors is within the level of those skilled in the art.

Hereinafter, the configuration and effects of the present invention will be more specifically described with reference to examples. However, the following examples are provided for illustrative purposes only to facilitate understanding of the present invention, and the scope and scope of the present invention are not limited thereby.

### EXAMPLE

### Example 1

### Experimental Materials and Methods

### (1-1) Preparation and Culture of Human T-Cells

The study protocol was reviewed and approved by the Institutional Review Board (IRB) of Seoul National University Hospital. Peripheral blood was drawn from healthy controls (HC) after obtaining written consent from study participants, and all experiments were performed according to approved guidelines. Peripheral blood mononuclear cells (PBMCs) were isolated from peripheral blood using density gradient centrifugation (Biocoll separation solution; BIOCHROM Inc., Cambridge, UK). Total CD4⁺ T cells, naive T cells and memory CD4⁺ T-cells were isolated in a negative selection manner from CD14⁺ monocyte depleted PBMCs using the MojoSort™ Human Total CD4⁺ T-Cells, CD4⁺ naive T-cells and CD4⁺ memory T-cell isolation kit (BioLegend, San Diego, Calif.). Purified T cells were cultured in RPMI 1640 medium (hereinafter, “complete RPMI 1640”) supplemented with 10% fetal bovine serum (FBS), 1% penicillin/streptomycin, and 1% L-glutamine. Cells were stimulated with anti-CD3/CD28-coated microbeads (1:10 ratio, Dynabeads T-Activator CD3/CD28, Thermo Fisher Scientific, Waltham, MA) in the presence of designated chemical inhibitors or reagents including 2-amino-2-norbornanecarboxylic acid (BCH), JPH203, acetate, MG132, VH298, cobalt chloride (CoCl₂), cycloheximid (CHX) (all provided by Sigma-Aldrich, St. Louis, MO), BCAT1 inhibitor 2 (Bi2: provided by Cayman Chemical, Ann Arbor, MI), 3-hydroxy-3-methylbutyric acid (HMB; provided by Thermo Fisher Scientific, Waltham, MA), and L-β-homoleucine (LβhL; provided by Santa Cruz Biotechnology, Heidelberg, Germany). In some experiments, a customized medium depleted of five essential amino acids (leucine, valine, isoleucine, phenylalanine, methionine) was used (Welgene, Gyeongsan, South Korea). All amino acids used (L-leucine, L-valine, L-isoleucine, L-phenylalanine, L-methionine) were purchased from Sigma-Aldrich.

### (1-2) Quantitative RT-PCR

Total RNA was prepared using TRIzol reagent (Life Technologies, Grand Island, NY), followed by cDNA synthesis (Promega, Madison, WI), followed by real-time quantitative RT-PCR with a CFX system (Bio-Rad, Hercules, CA) using SensiFAST SYBR® Lo-ROX (Bioline, London. UK). The sequences of the primers used in this study are shown in Table 1. Gene expression levels were normalized for ACTINB expression using the comparative CT method (ΔΔCT).

TABLE 1
Primers used for qPCR

| Gene name | Primer sequence (5′-3′) | SEQ ID NO. |
|---|---|---|
| human BCAT1 | Forward: AAGATGGGAGGGAATTACGG | 1 |
| | Reverse: TGGAGGAGTTGCCAGTTCTT | 2 |
| human BCAT2 | Forward: TTCCAGAAGGAGCTGAAGGA | 3 |
| | Reverse: CGCTGGCCTTTTATTTCGTA | 4 |
| human SLC7A5 | Forward: GAAGGCACCAAACTGGATGT | 5 |
| | Reverse: GAAGTAGGCCAGGTTGGTCA | 6 |
| human SLC3A2 | Forward: CCAGAAGGATGATGTCGCTCAG | 7 |
| | Reverse: GAGTAAGGTCCAGAATGACACG | 8 |
| human SLC7A8 | Forward: GACAGATAGTCCTTCGCTGGAAG | 9 |
| | Reverse: TCTGACCACAGGCTGAAGACCA | 10 |
| human SLC25A44 | Forward: GAGGCTATGTGGCTTCACTGCT | 11 |
| | Reverse: GACAATGTGAGGGCACTCCTTAG | 12 |
| human HPD | Forward: CCCTGGAACAAAGAGATGGGCGAT | 13 |
| | Reverse: GATTTTGGCGCCCCGTTCCC | 14 |
| human HPDL | Forward: CGAAGTCCCCAACCACAAGT | 15 |
| | Reverse: CGCCCTCGTTCACCAAAAAG | 16 |
| human BCKDK | Forward: TCCCCTTCATCCCTATGCCA | 17 |
| | Reverse: CAGAGACCCACCGAGGTACT | 18 |
| human HIF1A | Forward: CCATTAGAAAGCAGTTCCGC | 19 |
| | Reverse: TGGGTAGGAGATGGAGATGC | 20 |
| human SLC2A1 | Forward: TTGCAGGCTTCTCCAACTGGAC | 21 |
| | Reverse: CAGAACCAGGAGCACAGTGAAG | 22 |
| human LDHA | Forward: GGATCTCCAACATGGCAGCCTT | 23 |
| | Reverse: AGACGGCTTTCTCCCTCTTGCT | 24 |
| human PGK1 | Forward: CCGCTTTCATGTGGAGGAAGAAG | 25 |
| | Reverse: CTCTGTGAGCAGTGCCAAAAGC | 26 |
| human VGFA | Forward: TTGCCTTGCTGCTCTACCTCCA | 27 |
| | Reverse: GATGGCAGTAGCTGCGCTGATA | 28 |
| human IL-17A | Forward: ACTACAACCGATCCACCTCAC | 29 |
| | Reverse: ACTTTGCCTCCCAGATCACAG | 30 |
| human RORC | Forward: TTTTCCGAGGATGAGATTGC | 31 |
| | Reverse: CTTTCCACATGCTGGCTACA | 32 |
| human IFNG | Forward: TGACCAGAGCATCCAAAAGA | 33 |
| | Reverse: CTCTTCGACCTCGAAACAGC | 34 |
| human TBX21 | Forward: ATTGCCGTGACTGCCTACCAGA | 35 |
| | Reverse: GGAATTGACAGTTGGGTCCAGG | 36 |
| human BACTIN | Forward: GGACTTCGAGCAAGAGATGG | 37 |
| | Reverse: AGCACTGTGTTGGCGTACAG | 38 |
| mouse SLC7A5 | Forward: GGTCTCTGTTCACGTCCTCAAG | 39 |
| | Reverse: GAACACCAGTGATGGCACAGGT | 40 |
| mouse BCAT1 | Forward: CTGCCTCTGTTTTGCACTACGC | 41 |
| | Reverse: TCCTCACAGCAGATCGGCACAT | 42 |
| mouse BACTIN | Forward: AGCCATGTACGTAGCCATCC | 43 |
| | Reverse: CTCTCAGCTGTGGTGGTGAA | 44 |

### (1-3) Enzyme Linked Immunosorbent Assay (ELISA)

IL-17A and IFN-γ amounts in culture supernatants were quantified using commercially available human ELISA kits (IL-17A ELISA kit by eBioscience and human IFN-γ ELISA MAX Deluxe by BioLegend) according to the manufacturer's instructions. Light density was measured using an Infinite M200 instrument (Tecan, Männedorf, Switzerland).

### (1-4) Immunoblot Analysis

Cells were cultured in radioimmunoprecipitation assay (RIPA) buffer (150 mM NaCl, 10 mM Na2HPO4, pH 7.2, 1% Nonidet P-40, 0.5% deoxycholate) containing phenylmethylsulfonyl fluoride (PMSF; Millipore Sigma, Burlington, MA), EDTA, protease and phosphatase inhibitor cocktail (Thermo Fisher Scientific) to extract total protein. Proteins were separated on 8-10% SDS-polyacrylamide gels and blotted onto polyvinylidene difluoride (PVDF) membranes (Bio-Rad, Hercules, CA) followed by 5% BSA in Tris buffered saline (TBS) with 0.1% Tween 20 and blocked for 1 hour. Membranes were cultured overnight with anti-human SLC7A5, anti-BCAT, anti-phospho-p70S6K, anti-p70S6K, anti-HIF-1α (all provided by Cell Signaling Technology, Danvers, MA) primary antibodies at 4° C., followed by culture with HRP-conjugated secondary antibodies for 1 hour at room temperature. Membranes were developed using SuperSignal West Femto Maximum Sensitivity Substrate or SuperSignal West Pico PLUS Chemiluminescent Substrate system (Thermo Fisher Scientific).

### (1-5) Flow Cytometry

To analyze T cell proliferative capacity, purified isolated human CD4+ T cells were labeled with 1 μM carboxyfluorescein succinimidyl ester (CFSE; Invitrogen, Waltham, MA, USA) and then stimulated with anti-CD3/CD28 antibody coated microbeads for 4 days at 37° C. Cells were stained with 7AAD (BD Biosciences) for 10 minutes to distinguish dead cells and the extent of CFSE dilution in 7AAD-cells was measured by BD LSRFortessa to analyze proliferative capacity. Antibodies listed in Table 2 and previously known BD LSRFortessa cell analyzers were used.

TABLE 2
Antibodies used in flow cytometry

| Description | Source | Identifiers |
|---|---|---|
| Anti-mouse CD45 FITC (Rat monoclonal) | BD Bioscience | 553080 |
| Anti-mouse CD3e APC-Cy7 (Hamster | BD Bioscience | 557596 |
| monoclonal) | | |
| Anti-mouse CD4 PE (Rat monoclonal) | BD Bioscience | 561837 |
| Anti-mouse CD11b V450 (Rat monoclonal) | BD Bioscience | 560455 |
| Anti-mouse CD4 PerCP (Rat monoclonal) | BD Bioscience | 553052 |
| Anti-mouse F4/80 PE-Cy7 (Rat monoclonal) | Invitrogen | 25-4801- |
| | | 82 |
| Anti-mouse IL-17A Brilliant Violet 421(Rat | BioLegend | 506925 |
| monoclonal) | | |
| Anti-mouse IFN-γ APC (Rat monoclonal) | Invitrogen | 17-7311- |
| | | 82 |
| Anti-human/mouse HIF-1α APC (Mouse | R&D Systems | IC1935A |
| monoclonal) | | |

### (1-6) ³H-Amino Acid Absorption Analysis

After the culture medium was removed, the cells were cultured in HBSS for 10 minutes. ³H-leucine or ³H-methionine (Perkin Elmer, Waltham, MA) uptake assays were initiated by culturing cells for 15 min in HBSS with 0.5-1 μCi. Cells were separated with 1 M NaOH after three washes with ice-cold HBSS. Radioactivity was measured using a beta scintillation counter MicroBeta® (Perkin Elmer).

### (1-7) Lentivirus Production and Transduction of Human CD4⁺ Memory T-Cells

BCATc, HPD or HPDL human shRNA plasmids (Origene, Rockville, MD: Cat #TL314498 for BCATc, TL312348 for HPD, TL304319 for HPDL, TR30021 for control) including the GFP reporter gene were used to inhibit BCATc, HPD and HPDL expression in human CD4⁺ memory T-cells. Lentivirus was produced by transfection of lentivirus vectors and psPAX2 (#12260) and pMD2.G (#12259) (both purchased from Addgene, Watertown, MA) expression vectors into 293FT-cells using Fugene (Promega, Madison, WI). Lentiviral particles were collected 48 and 72 hours post transfection, filtered through a 0.45 μm syringe filter (Millipore), concentrated using Peg-it solution (System Biosciences, Palo Alto, CA), and then titrated in 293FT-cells. Purified CD4⁺ memory T-cells for lentiviral transduction were activated with anti-CD3/CD28 coated microbeads and transduced with lentiviral vectors expressing scrambled control or target shRNAs at a multiplicity of infection of 10 in the presence of 8 mg/ml polybrene (Sigma). After 24 hours, shRNA⁺ cells expressing GFP were sorted by BD FACSAria™ III cell sorter (BD Bioscience) and activated for 3 more days with microbeads coated with anti-CD3/CD28.

### (1-8) Cholesterol Analysis

Total cholesterol amount in cultured cells was determined using a fluorometric Amplex™ Red Cholesterol Assay Kit (Thermo Fisher Scientific) according to the manufacturer's instructions.

### (1-9) ScRNA-Seq Analysis

Freshly purified CD4⁺ memory T-cells were stimulated for 72 hours with anti-CD3/CD28 coated microbeads in the absence or presence of Bi2. Samples from four different donors were multiplexed for 30 minutes at 4° C. with a hashtag oligo. Totalseq™ anti-human hashtag antibody (Biolegend, USA) was then used to perform in three experimental conditions (TCR-stimulated cells with Bi2, TCR-stimulated cells without Bi2, and cells without TCR stimulation). Tagged cells were pooled and loaded into a Chromium system (10× Genomics, Pleasanton, CA) and encapsulated into a single droplet targeting about 40,000 cells for each GEM (Gel Beads-in-emulsion). Chromium Single Cell 5′ Kit (10× Genomics) was used to generate scRNA-seq and TCR libraries according to the manufacturer's instructions. Demultiplexing and read alignment to the bound human genome was performed using Cell Ranger (v6.1.2).

### (1-10) EAE Induction

Female C57BL/6 mice (12 weeks old) used for EAE induction were purchased from KOATECH (Pyeongtaek, Gyeonggi-do, Korea). All mice were bred and managed in a pathogen-free facility at Seoul National University (SNU) Medical School. All experiments were approved by the SNU IACUC. Mice were injected intraperitoneally with 10 mg/kg BCAT1 inhibitor 2 (Bi2) dissolved in 200 μl of sterile PBS three times a week until 14 days after immunization. To induce EAE, mice were injected subcutaneously on both sides with a total of 200 μg of MOG 35-55 peptide (GeneScript, Piscataway, NJ) emulsified in CFA (BD Biosciences, San Jose, CA), followed by intraperitoneal injection of 250 ng of pertussis toxin (PTX) (List Biological Labs, Campbell, CA) after 4 and 24 hours. Clinical scores of EAE mice were evaluated daily: 0 point, if there is no obvious change in motor function, 0.5 point, if the tail tip is drooped, 1 point, if the tail is hanging down, 1.5 point, if there is tail and hind limb suppression, 2 point, if there is tail or hind limb weakness, 2.5 point, if the tail is hanging down and the hind limb is pulled, 3 point, if the tail and hind leg are fully paralyzed, 3.5 point, if the hind leg is fully paralyzed and the hind leg leans to one side of the body, 4 point, if the tail is hanging down and there is partial paralysis of the hind leg and the front leg, 4.5 point, if the hind and front legs are fully paralyzed and cannot move in the cage. At the end of the experiment mice were euthanized and immune cells were isolated from the spinal cord. Spinal cords were chopped and treated with 1 mg/ml collagenase D (Sigma-Aldrich) and 50 μg/ml DNase I (Thermo Fisher Scientific, Waltham, MA) at 37° C. for 30 minutes, followed by filtration through a 70 μm cell filter. After enzyme treatment, monocytes infiltrated into the central nervous system were isolated via Percoll (Cytiva, Upsala, Sweden) density gradient (30:70) centrifugation.

### (1-11) LC-MS Analysis of Intracellular Alpha-Ketoisocarproic Acid

A total of 1×10⁶ HepG2 cells were cooled with 200 μl of ice-cold 50% methanol dissolved in distilled water. After centrifugation the supernatant was transferred to a new tube and 200 μl of ice-cold 50% methanol was added to the remaining pellets. After stirring and centrifugation the supernatant was collected and combined with the previously collected supernatant. Samples were stored at −80° C. until analysis. Samples were thawed prior to analysis, filtered through a 0.2 μm syringe filter, and then transferred to LC-MS tubes.

Alpha-ketoisocarproic acid (α-KIC) levels were analyzed using liquid chromatography-orbitrap mass spectrometry coupling Thermo Fisher Scientific Vanquish™ UPLC system and Thermo Fishers Scientific Orbitrap Exploris™ 120. Separation was performed using an ACQUITY UPLC HSS T3 column (100 Å, 1.8 μm, 2.1 mm×100 mm). HPLC grade water was used as mobile phase A and HPLC grade methanol as mobile phase B. The column was kept at 40° C., the injection amount was 5 μl and the gradient was carried out at a flow rate of 0.3 ml min⁻¹. Samples were eluted with a linear slope (curve=5) consisting of 5-30% B for 0-3 minutes, 90% B for 3.5 minutes, 90% B in 3 minutes, 5% B in 7 minutes, and 5% B in 3 minutes as recommended in previous studies. A constant slope was maintained at 90% B for 3 minutes, 5% B for 7 minutes, and 5% B for 3 minutes for re-equilibration.

Mass spectrometry was performed with the following parameters. Spray voltage, 4000 V for positive mode, 3000 V for negative mode; Sheath gas flow rate, 40 L h⁻¹; aux gas flow rate, 10 L h⁻¹; sweep gas flow rate, 1 L h⁻¹; ion transfer tube temperature, 340° C.; evaporator temperature, 350° C. The α-KIC was specifically determined by a targeted single ion monitoring (tSIM) scan, followed by a data-dependent MS² (ddMS²) scan with a target mass filter. For both metabolites, the separation window was set to 2 (m/z), the Orbitrap resolution was set to 120,000 for tSIM and 15,000 for ddMS², the RF lens was set to 70%, and the scan range was set to 150-2000 (m/z) for mass scan and 40-150 (m/z) for ddMS². The collision energy was set to 20 eV for α-KIC. Definition ions were selected for identification as follows. α-KIC (129.0556>69.0346). Thermo Fisher Scientific FreeStyle program was used for peak detection and area calculation.

### (1-12) Statistical Analysis

The Mann-Whitney U test between the two groups was used for comparison between the two groups, and the Turkey post hoc test or two-way ANOVA was used for comparison of three or more groups. This analysis was performed using Prism 9 software (GraphPad Software Inc., La Jolla, CA) and was deemed statistically significant if the p value was less than 0.05.

### Example 2

### Up-Regulation of BCAT1 and SLC7A5 Expression by TCR-Stimulated CD4⁺ T-Cells

Accumulating evidence has shown that BCAA supplied by SLC7A5 plays an important role in metabolic rewiring in activated immune cells. Isoleucine uptake through SLC3A2, which forms a heterodimer with SLC7A5 in murine Treg cells, activates the mTORC1 pathway, thereby altering the metabolic state. Accumulation of BCAAs in CD8⁺ T-cells increases Glut1 expression, leading to increased glucose uptake, which promotes metabolic reprogramming and anti-tumor immunity. SLC7A5-mediated leucine influx in macrophages induces IL-10 production via mTORC1-induced glycolytic reprogramming upon LPS stimulation. BCAT1-regulated BCAA degradation in activated macrophages is associated with inflammatory disease and itaconate production in the TCA cycle.

Despite being essential for protein synthesis, an increase in BCAA is associated with systemic diseases, including cancer, heart failure, diabetes, and insulin resistance, which may be due to its involvement in the signaling role of the TCA pathway and metabolic pathways. For example, leucine activates mTORC1 in direct association with leucine sensors such as leucyl-tRNA synthetase or Sestrin2. Although less well known, leucine-derived metabolites also support metabolic rewiring in activated immune cells. Reprogramming of BCAA metabolism relies on altered expression and activity of metabolic enzymes (e.g., BCAT) and transporters (e.g, SLC7A5).

According to the present study, TCR stimulation increases the expression of BCAT and SLC7A5 in human CD4⁺ T-cells, allowing these proteins to play a key role in BCAA metabolism reprogramming. To examine the immunomodulatory role of BCAT in T-cell responses, we first examined BCAT expression in TCR-activated T-cells derived from healthy controls (HC). Cytoplasmic BCAT1 mRNA and protein expression was significantly increased in CD4⁺ T-cells upon TCR stimulation, and mitochondrial BCAT2 was also slightly upregulated in these cells (FIGS. 1A and 1B). Since BCAT1 promotes the reversible amine conversion of BCAA to become the first enzyme in the degradation pathway, we next sought to determine if TCR stimulation induces the expression of BCAA-specific transporters. Analysis of public RNA-Seq data (GSE140244) and confirmatory qPCR revealed that the expression of several amino acid transporters, including BCAA specific SLC7A5 and SLC3A2, in human CD4⁺ T-cells was dependent on TCR stimulation (FIGS. 1C and 1D). As shown in FIG. 1E, TCR stimulation predominantly upregulated SLC7A5 expression in human primary CD4⁺ T-cells but not in CD8⁺ T-cell under the same culture conditions (FIG. 2A). In addition, TCR-activated CD4⁺ T-cells efficiently absorbed ³H-labeled leucine, which is BCAA, and this absorption was greatly reduced in the presence of JPH203, a specific inhibitor of SLC7A5 (LAT1), or 2-amino-2-norbornanecarboxylic acid (BCH), a general inhibitor of LAT (FIG. 1F). TCR-activated CD4⁺ T-cells also mediated the uptake of methionine, which is mainly transported by SLC7A5, suggesting that TCR-induced SLC7A5 functions well (FIG. 2B). The results show that TCR stimulation upregulates the expression of SLC7A5 and BCAT1, increasing the uptake and metabolism of BCAA by human CD4⁺ T-cells.

### Example 3

### Effect of LC7A5-Mediated Leucine Inflow on CD4⁺ T-Cell Response

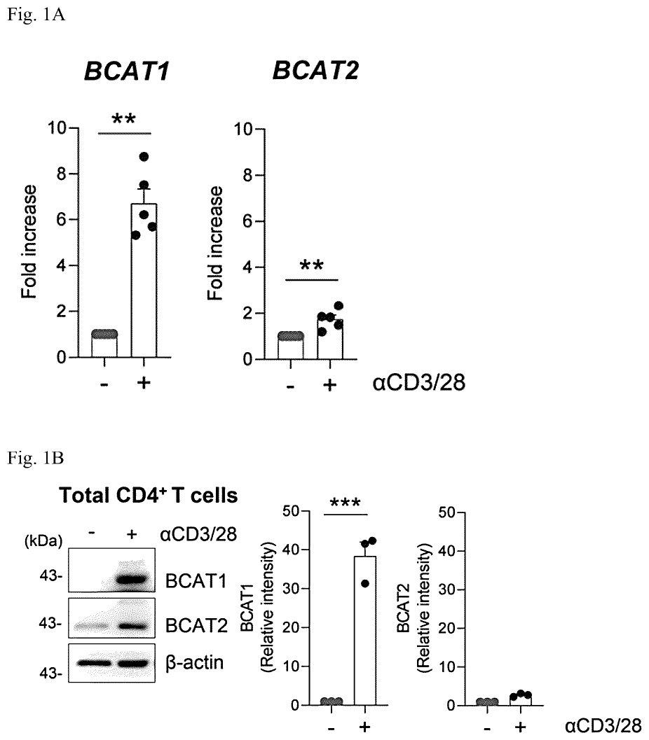

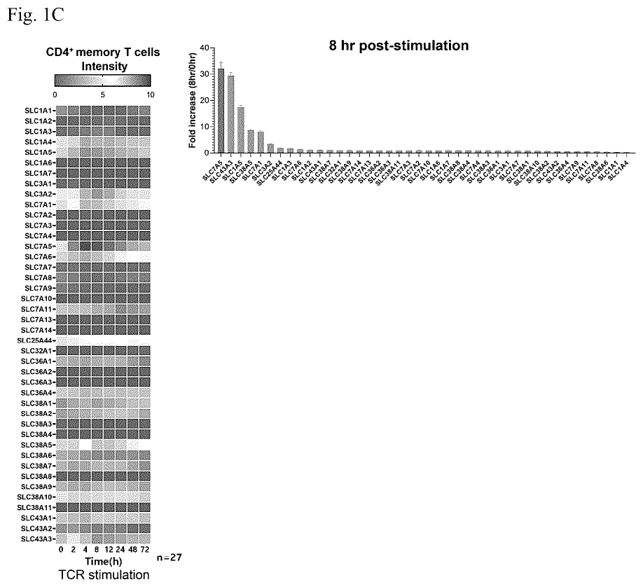

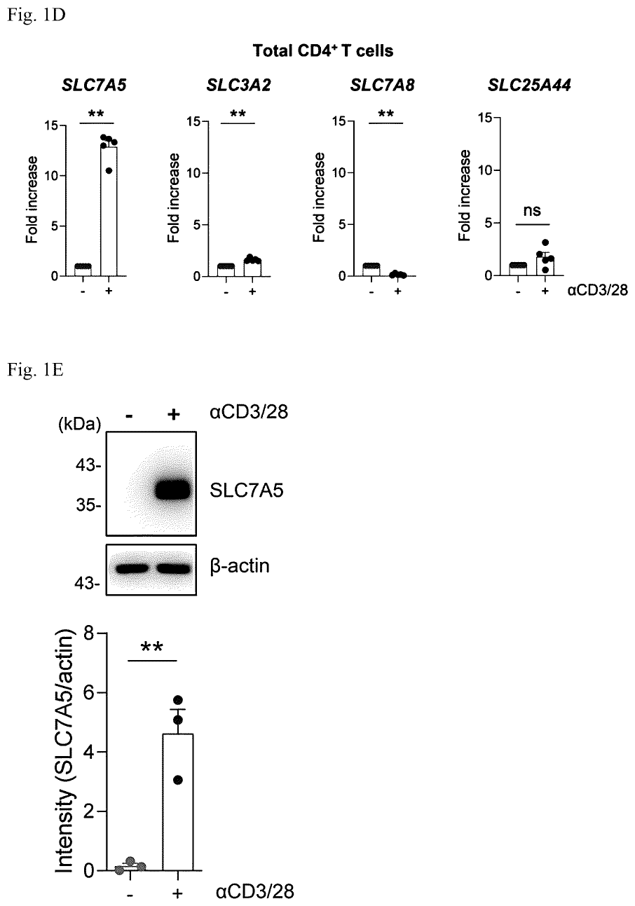

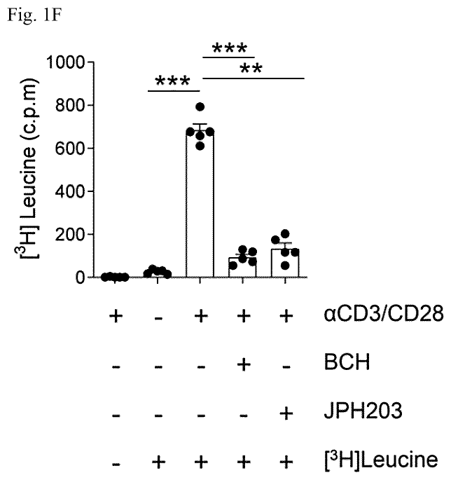

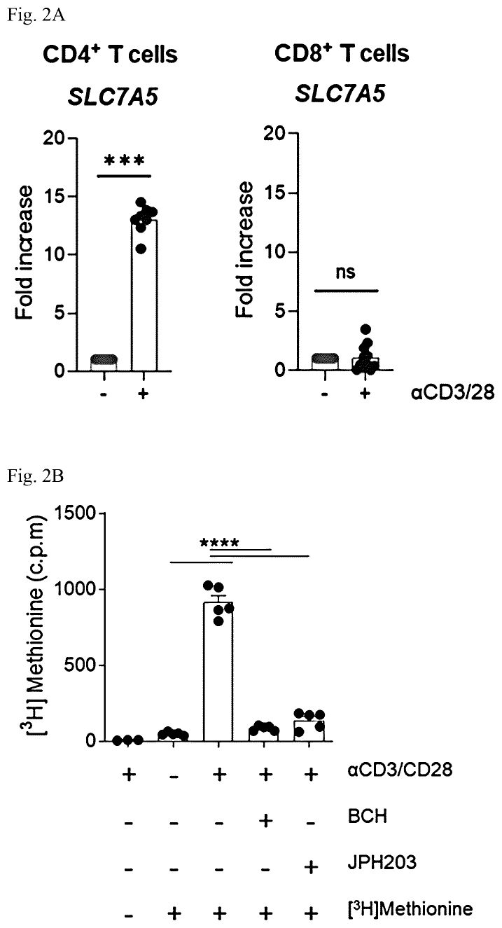

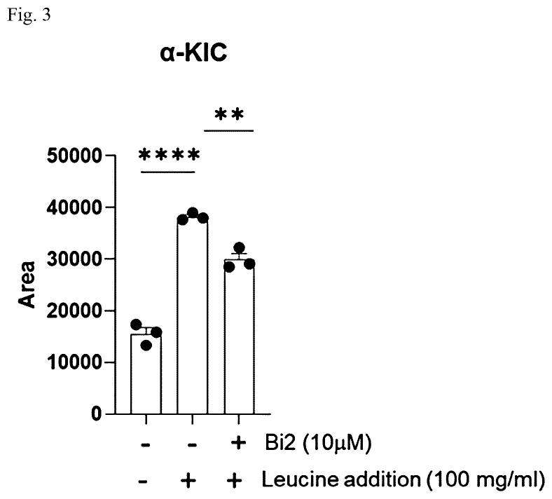

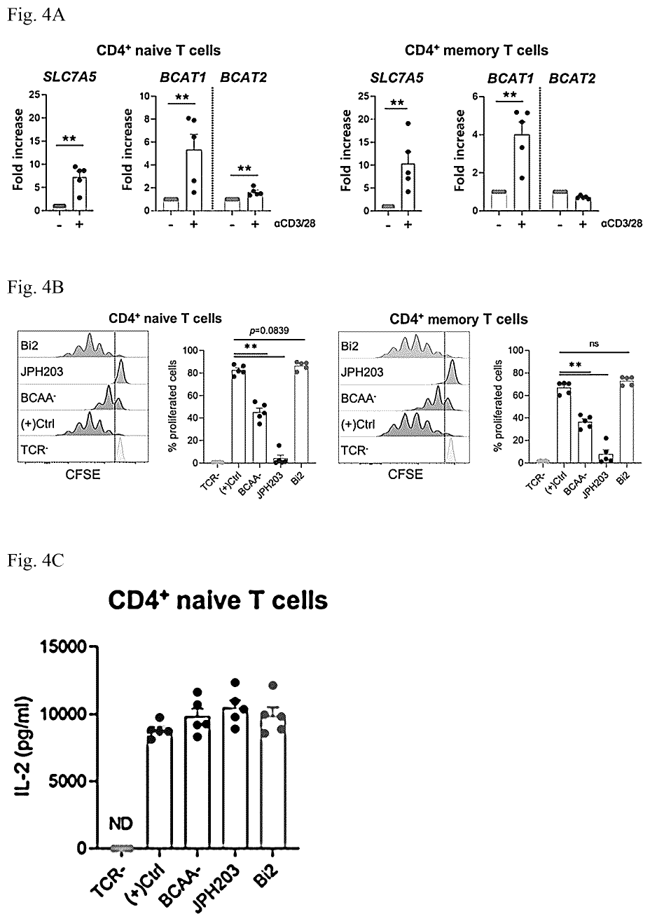

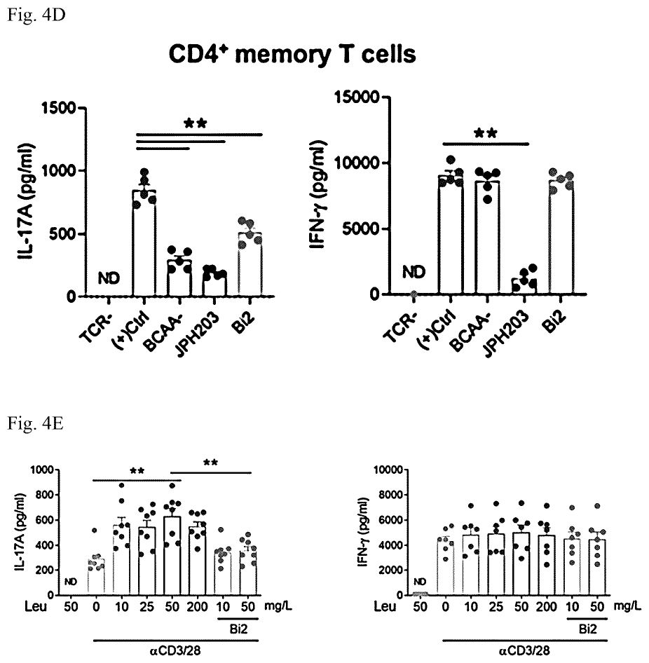

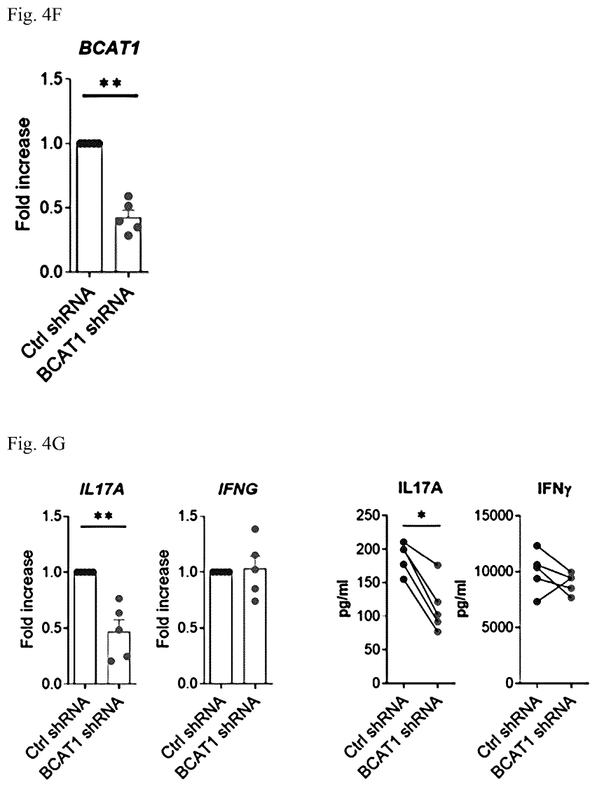

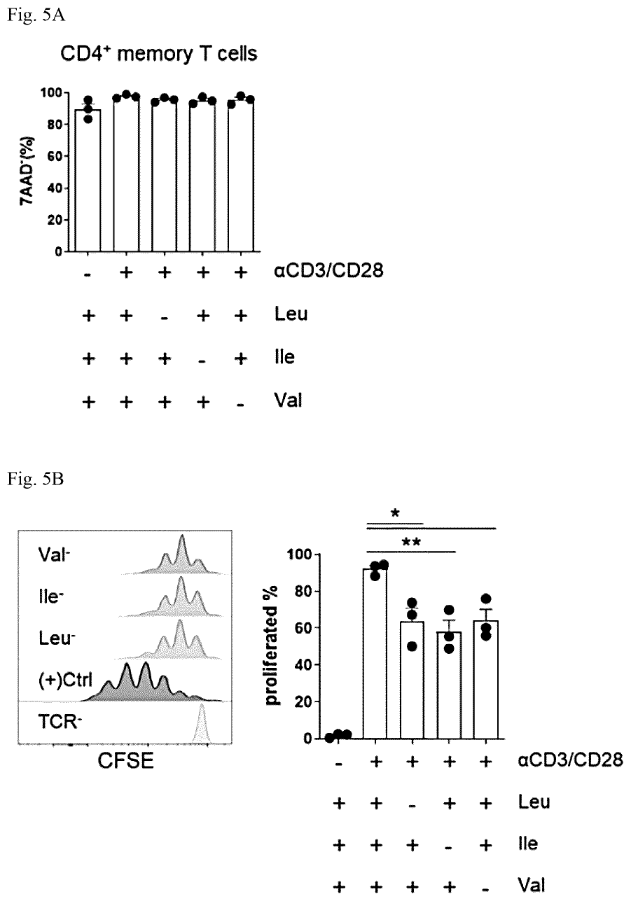

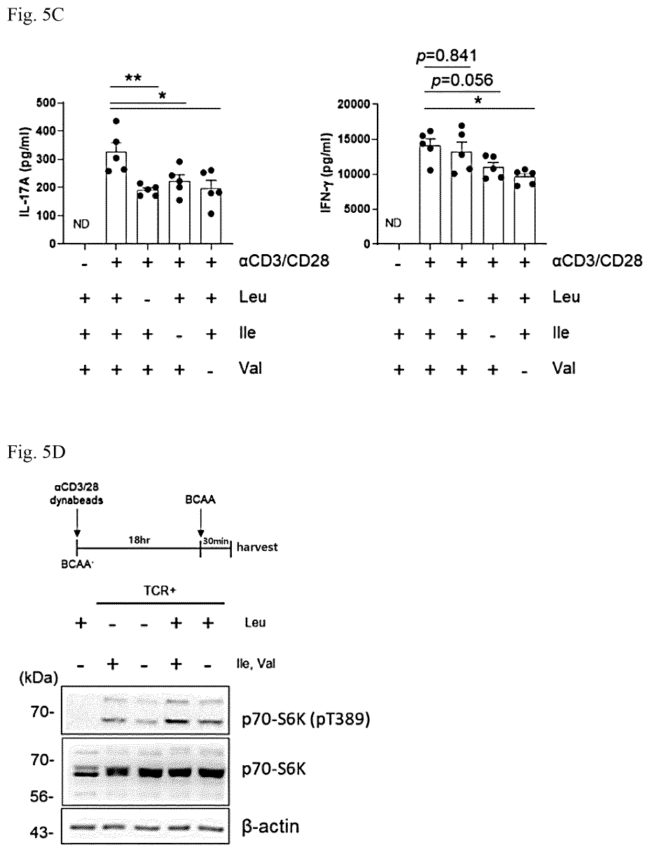

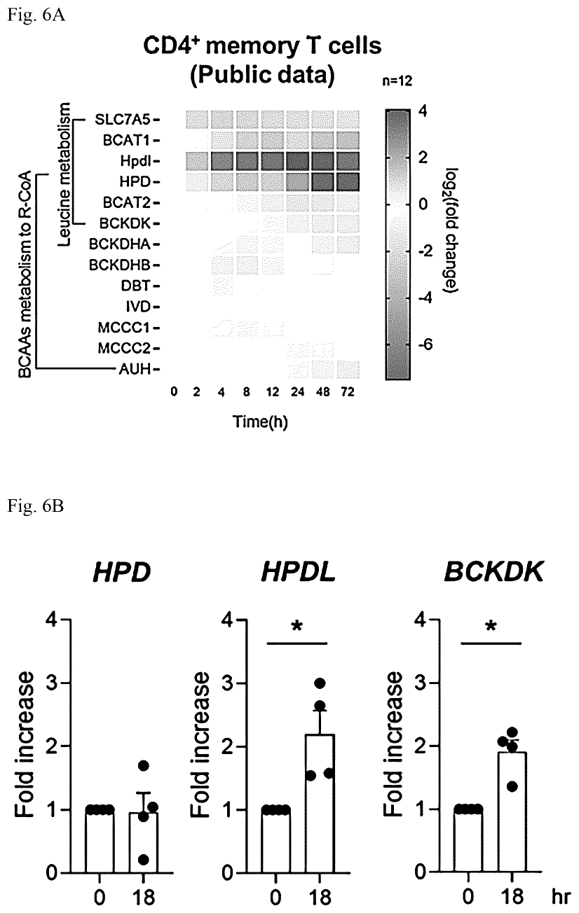

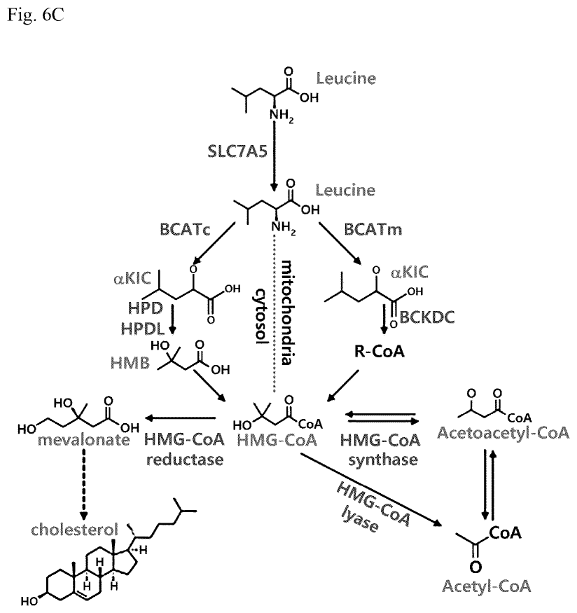

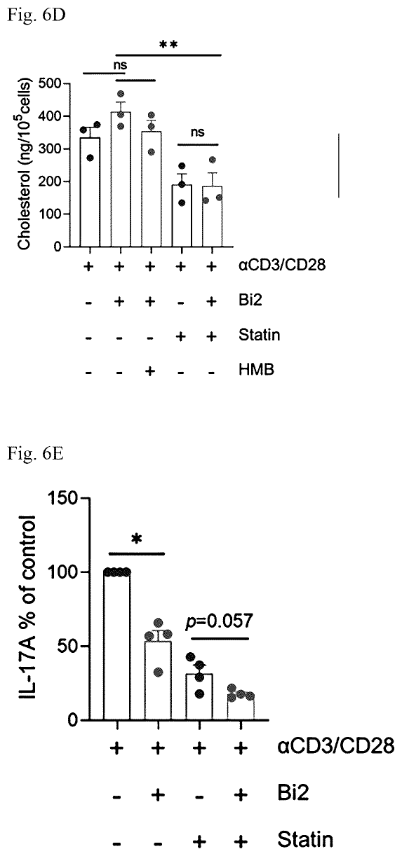

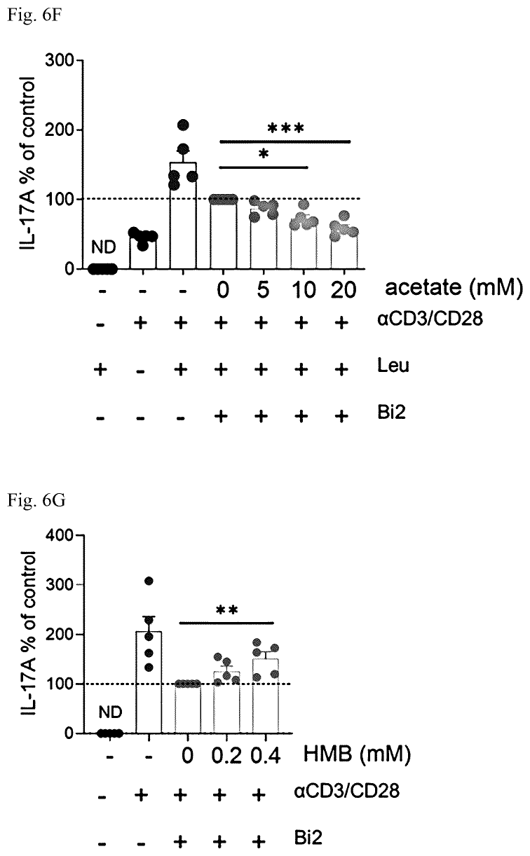

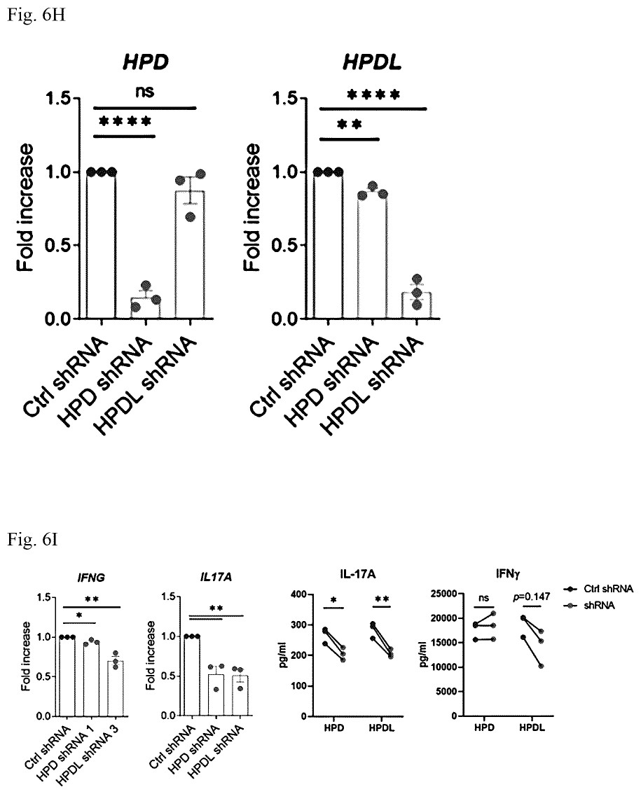

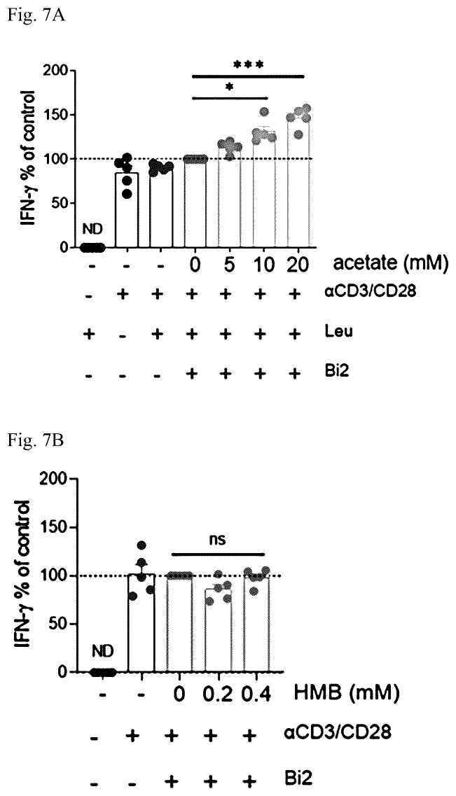

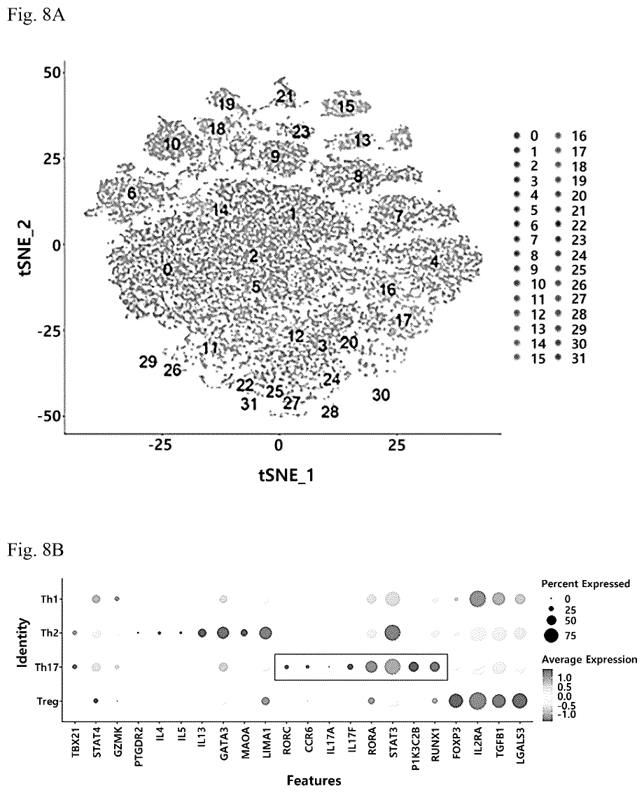

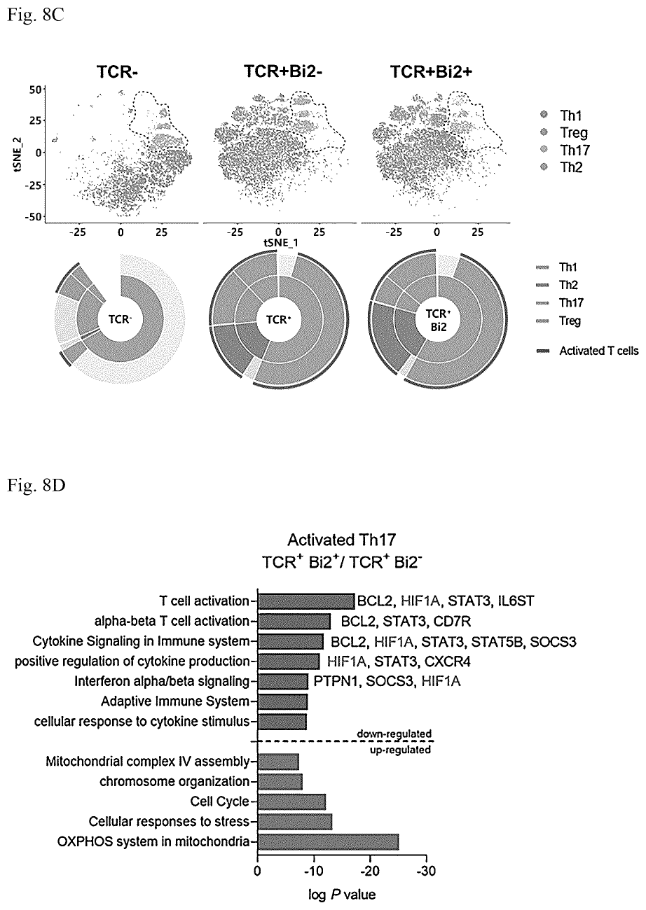

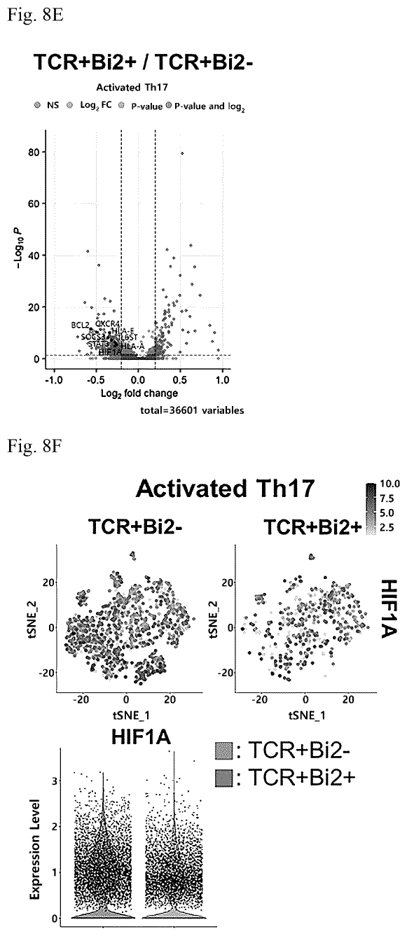

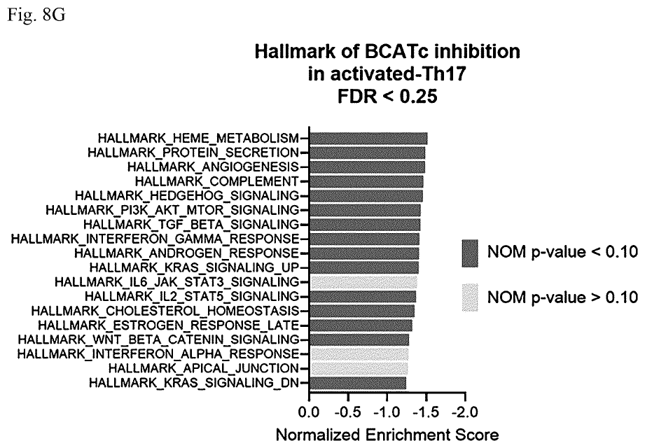

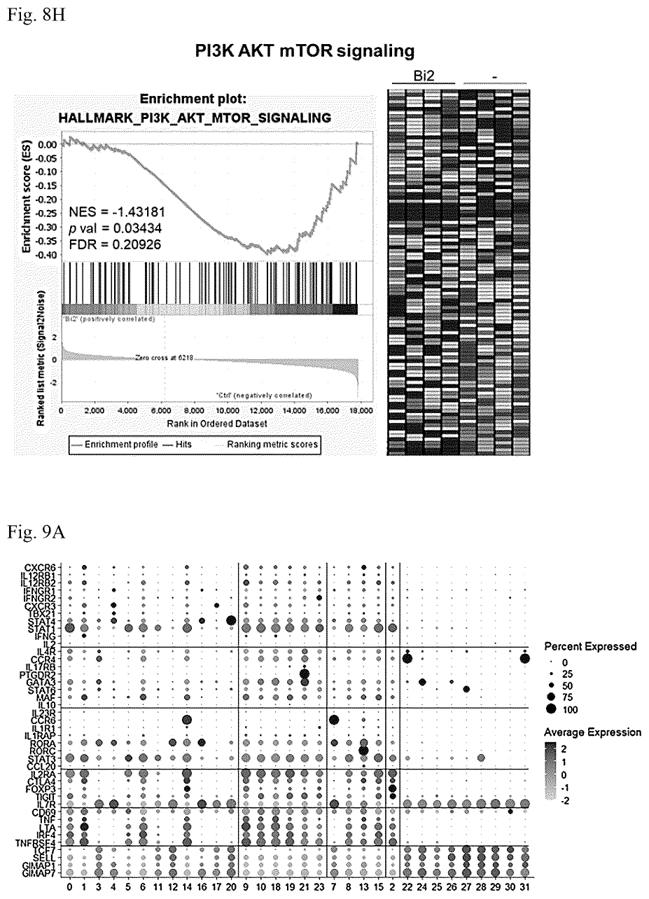

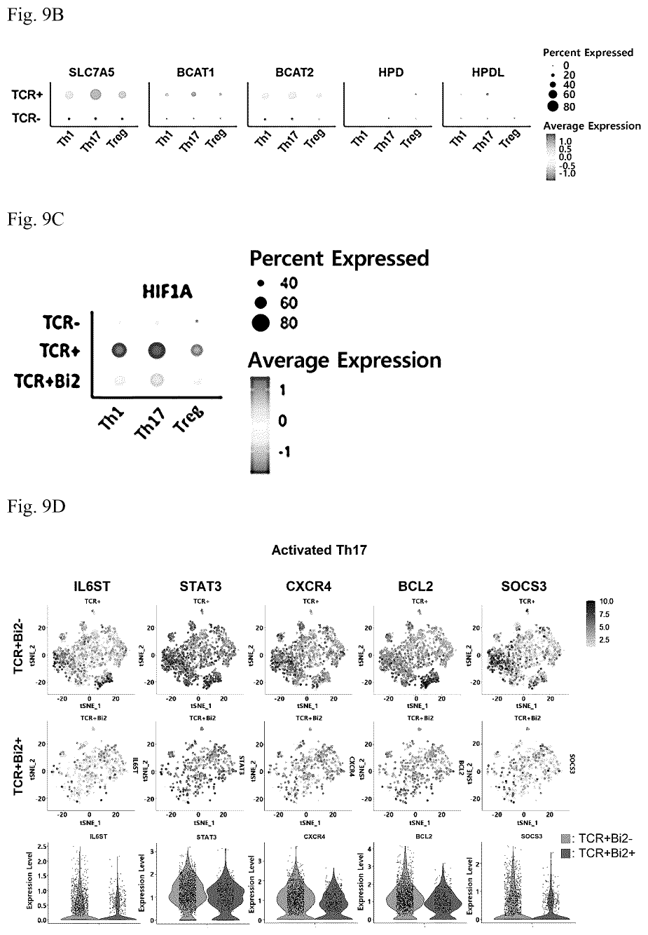

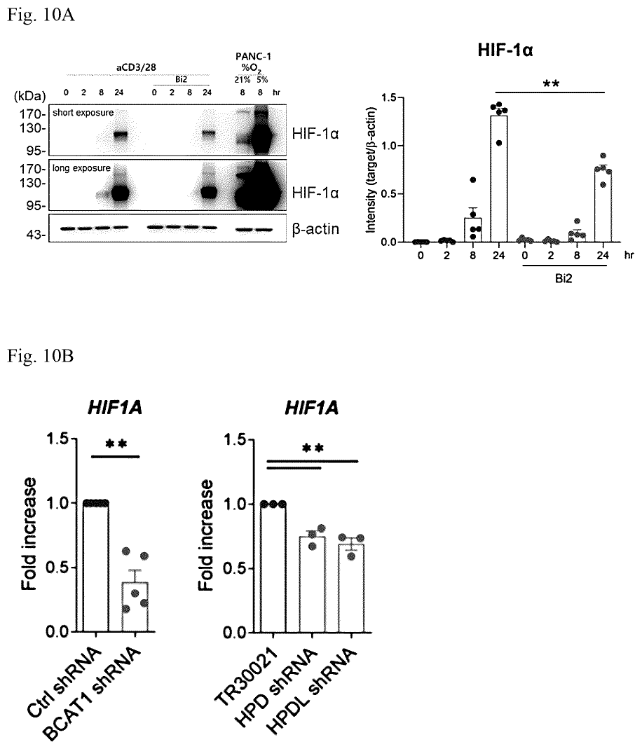

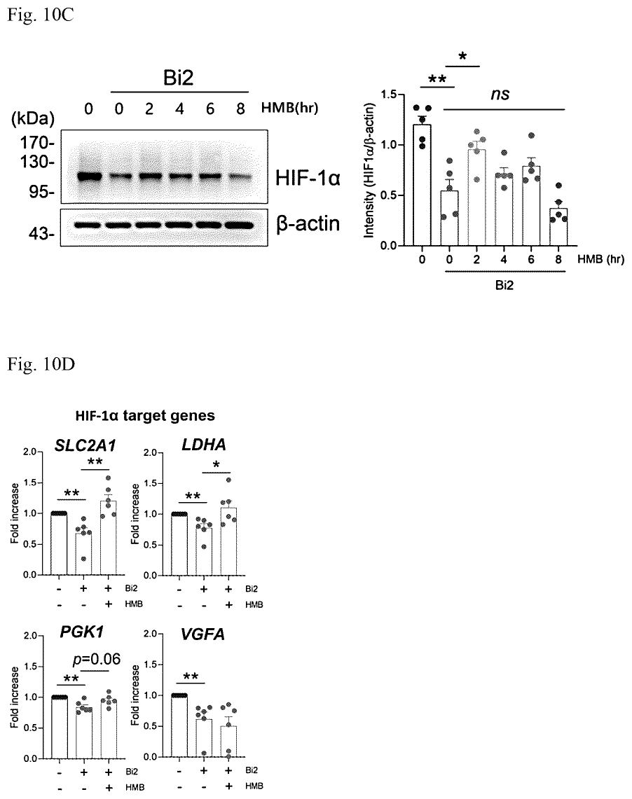

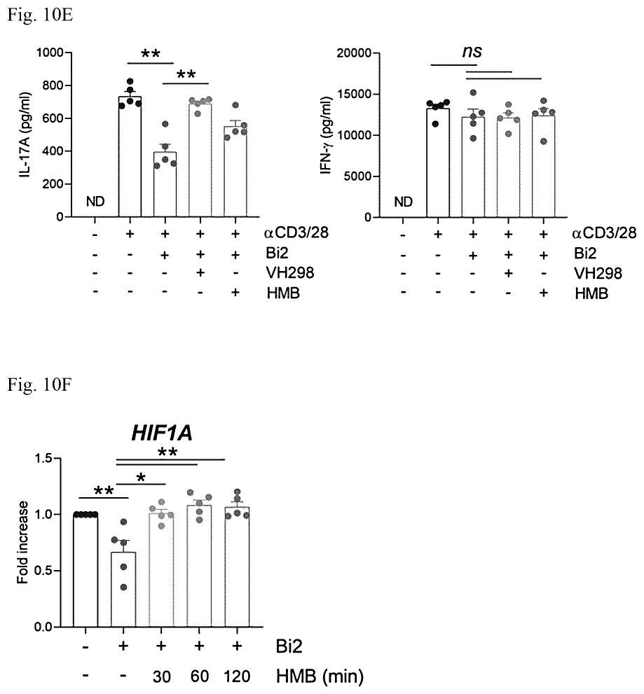

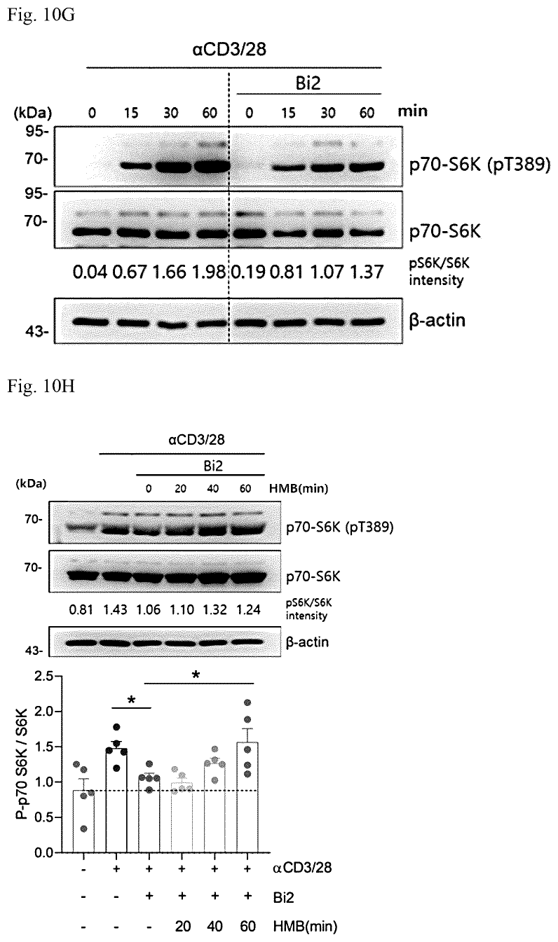

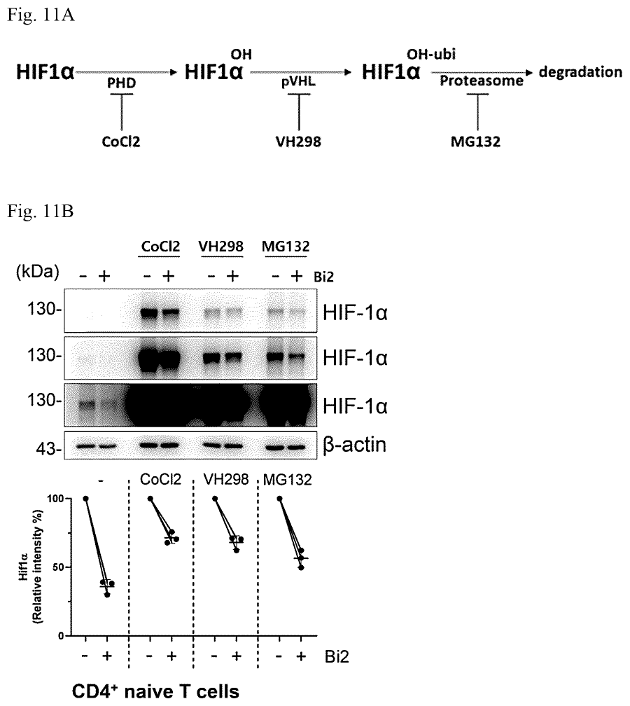

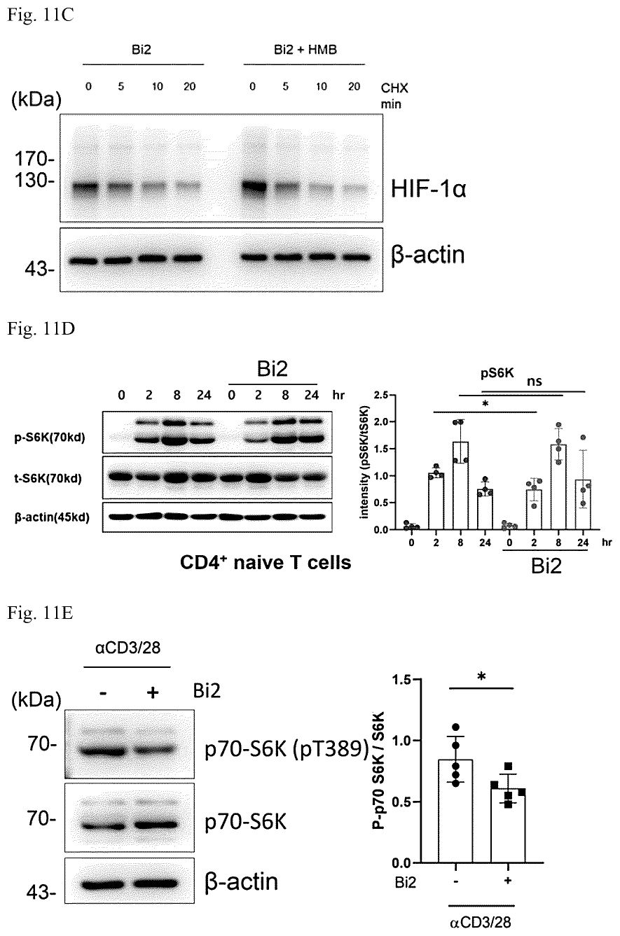

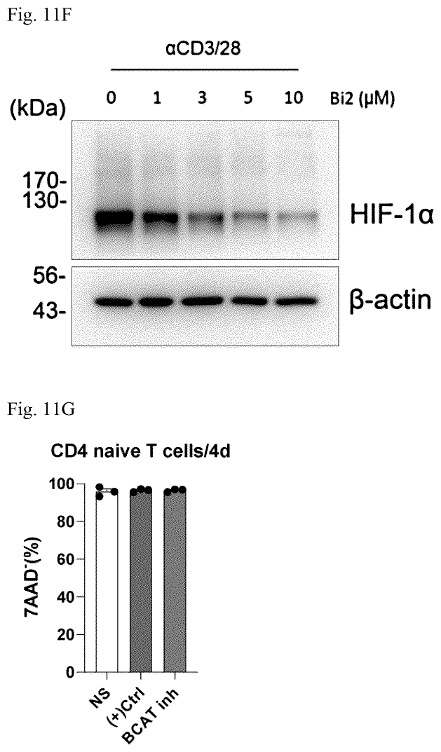

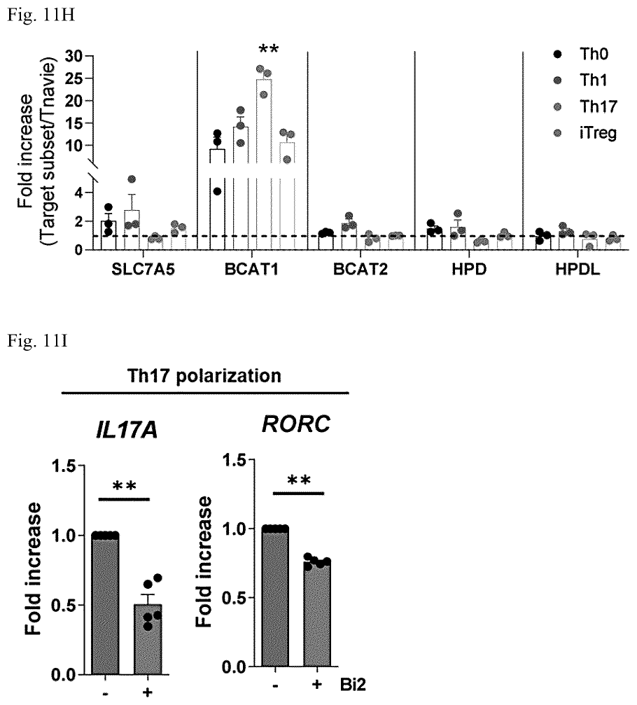

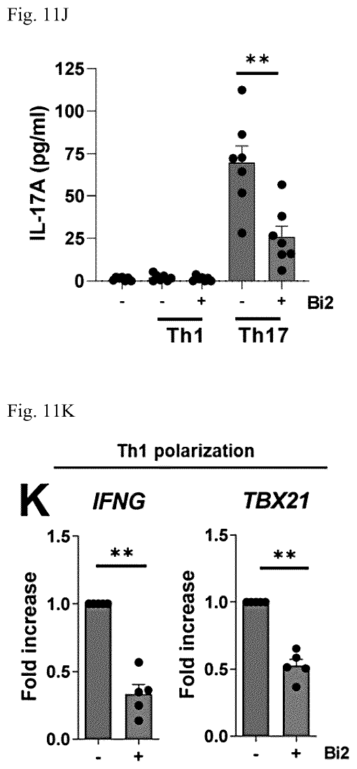

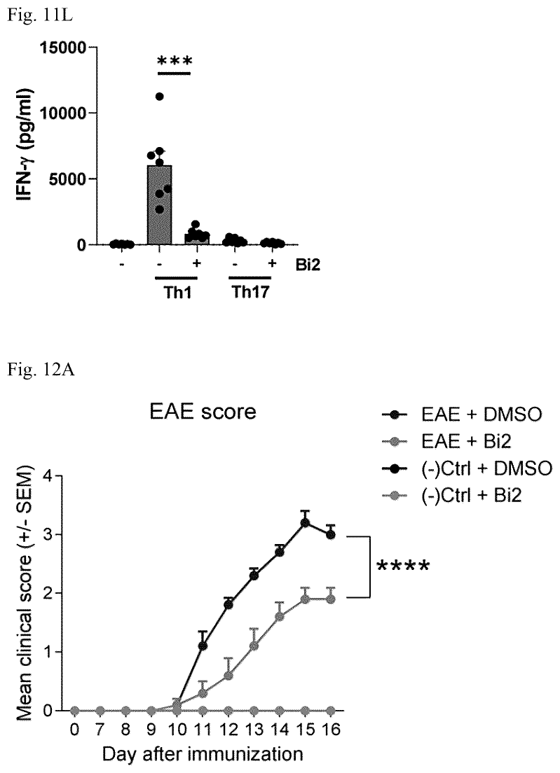

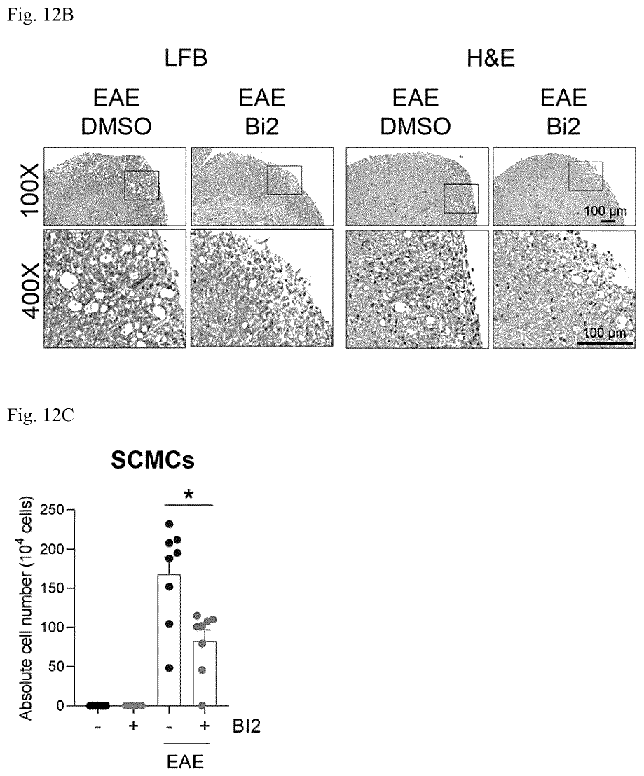

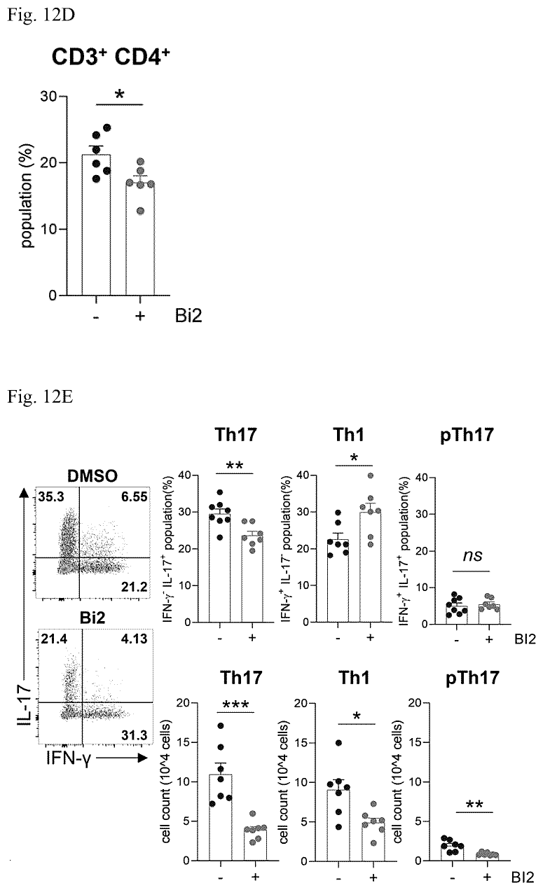

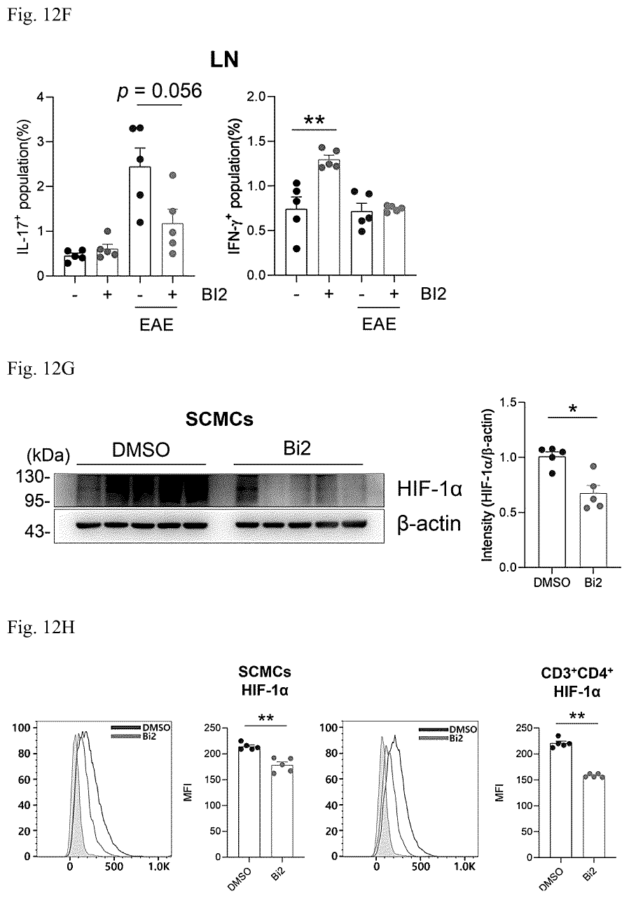

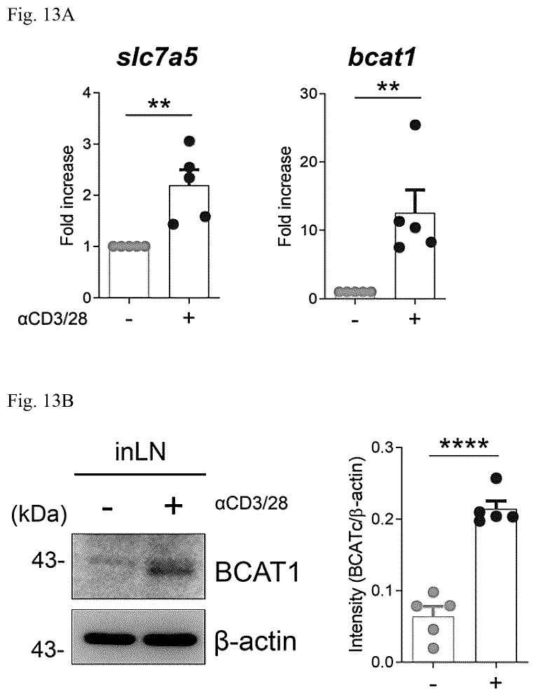

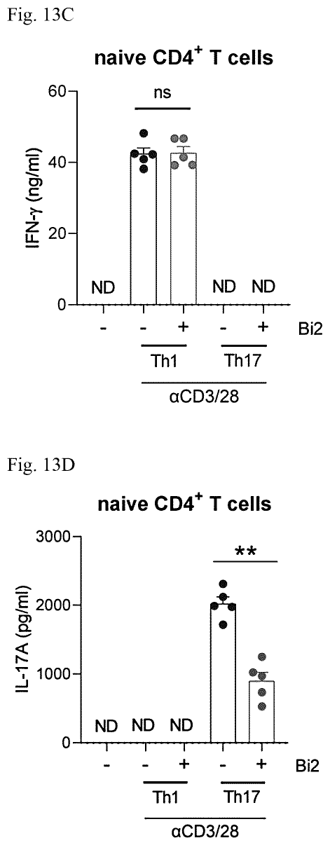

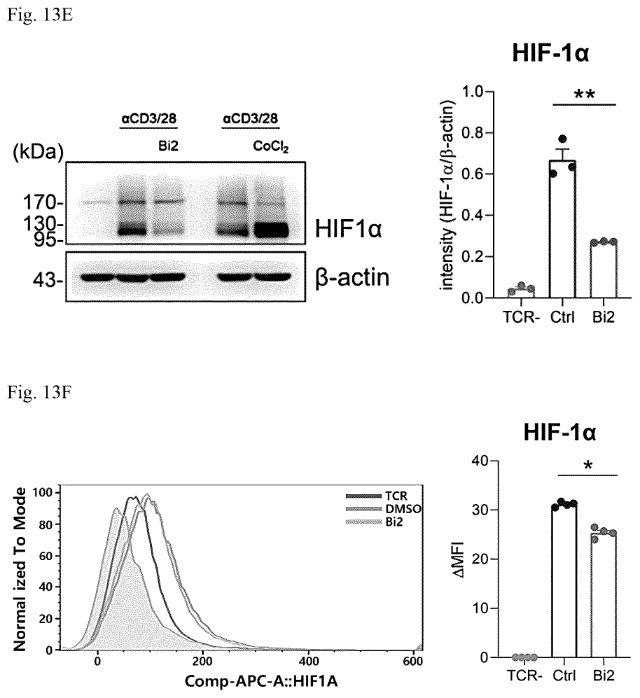

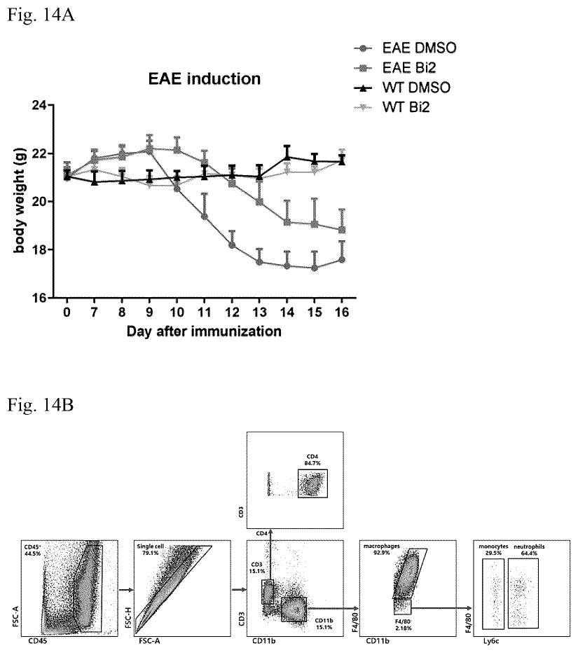

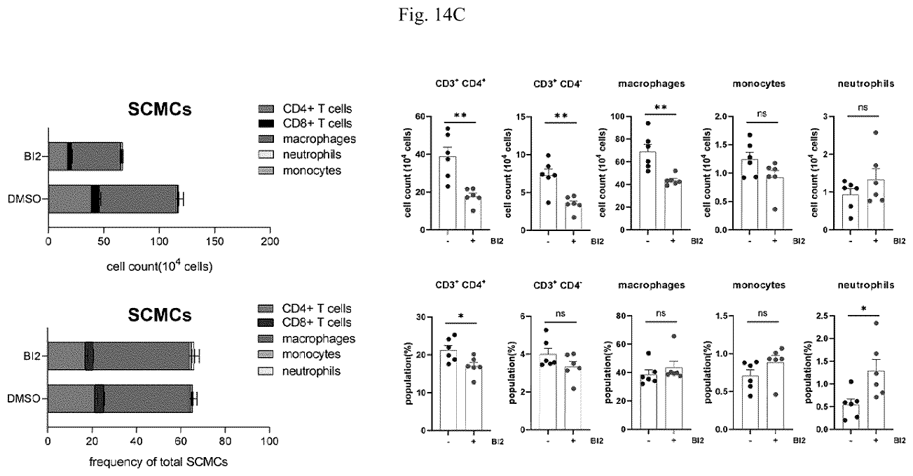

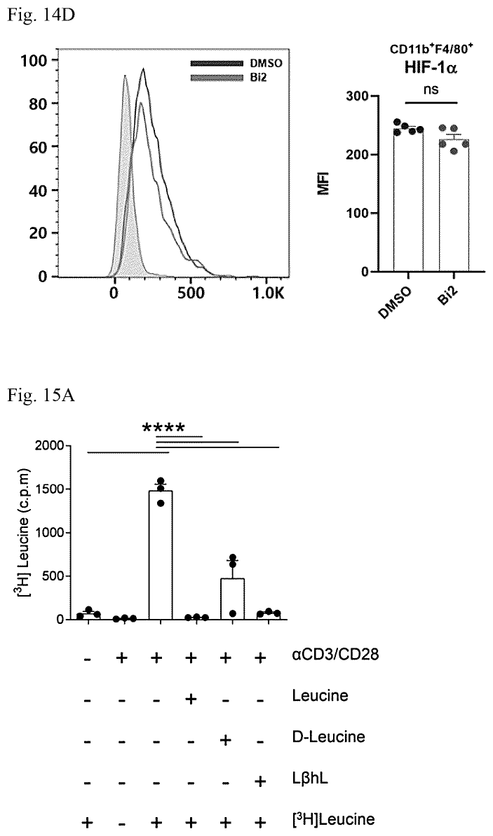

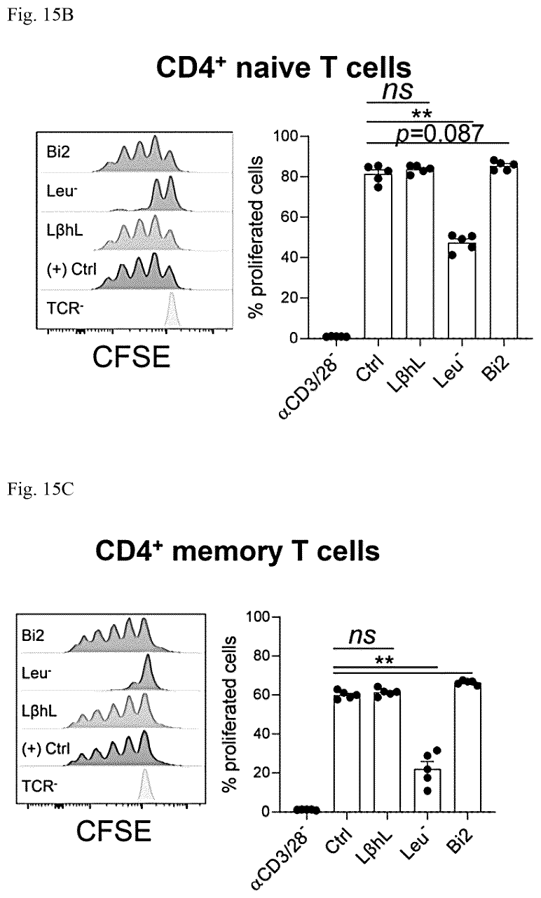

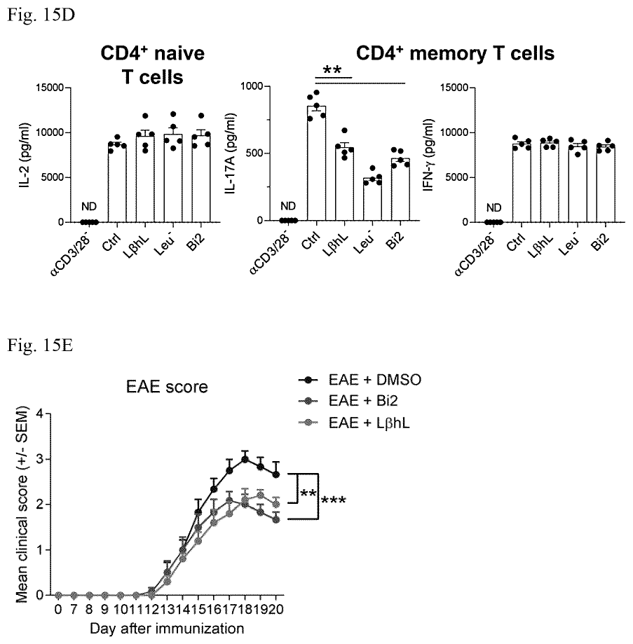

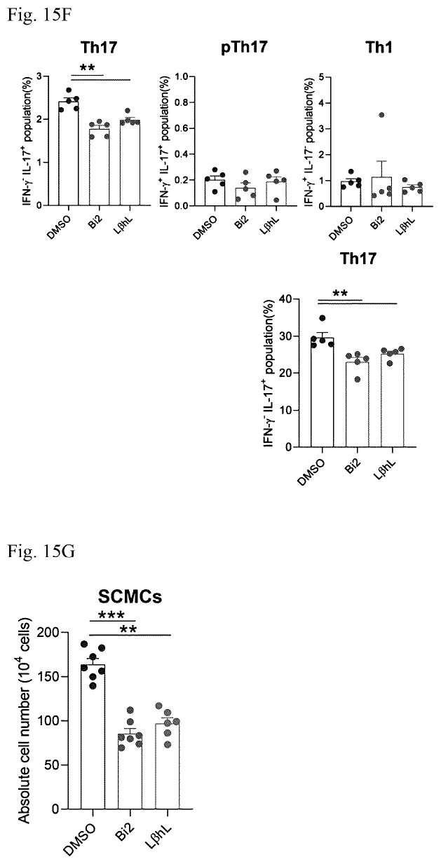

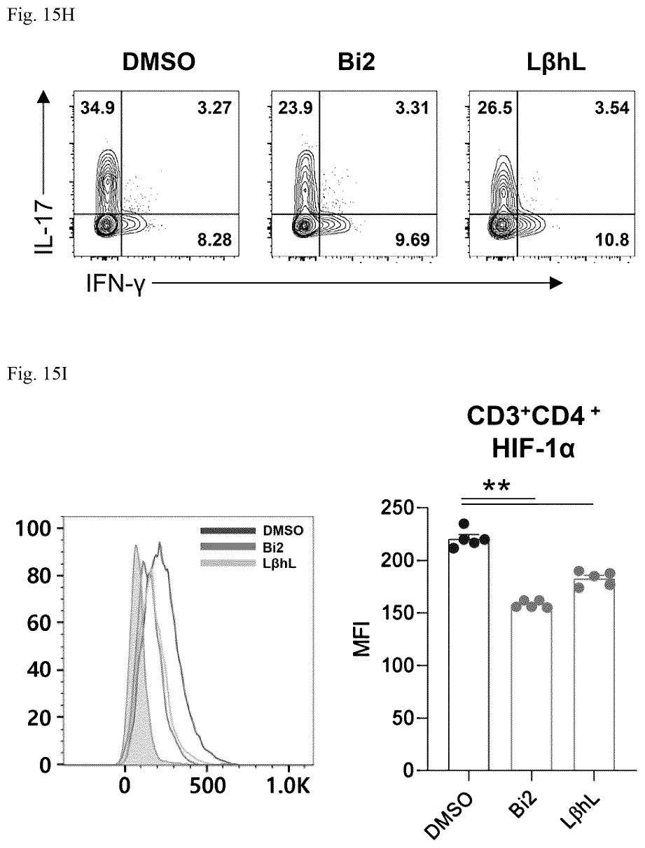

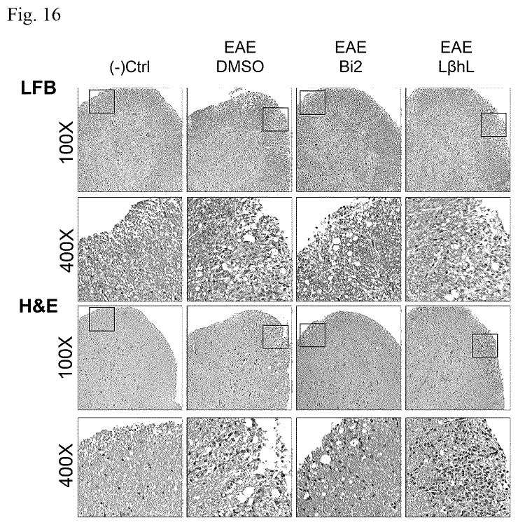

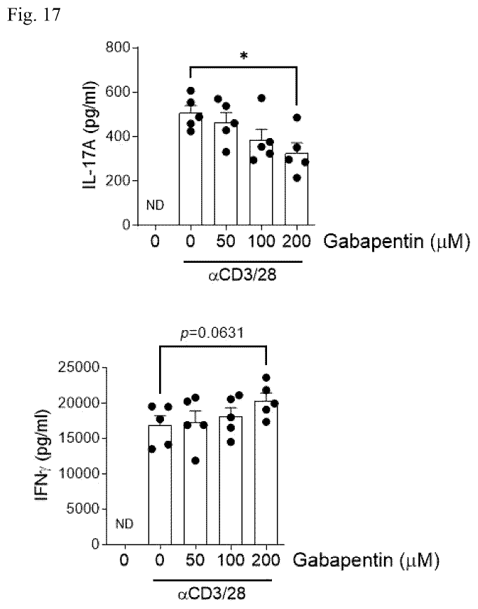

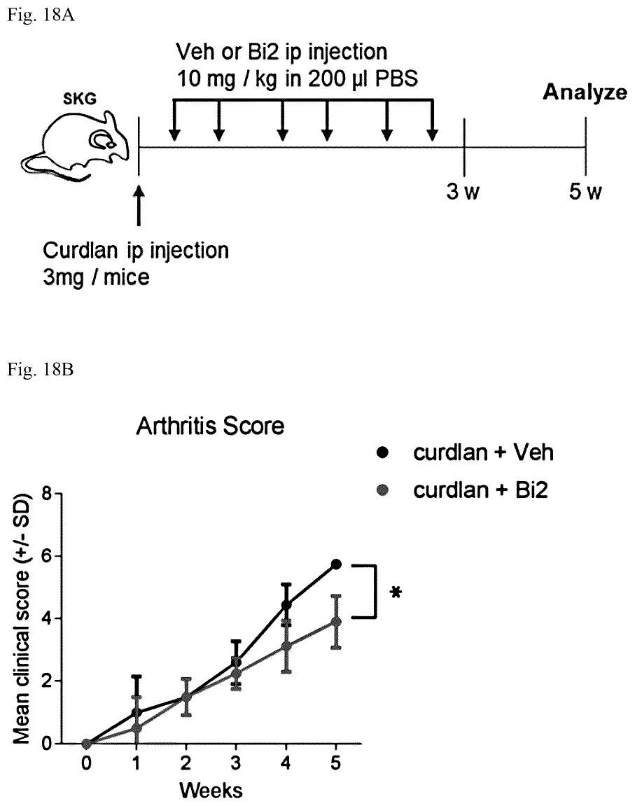

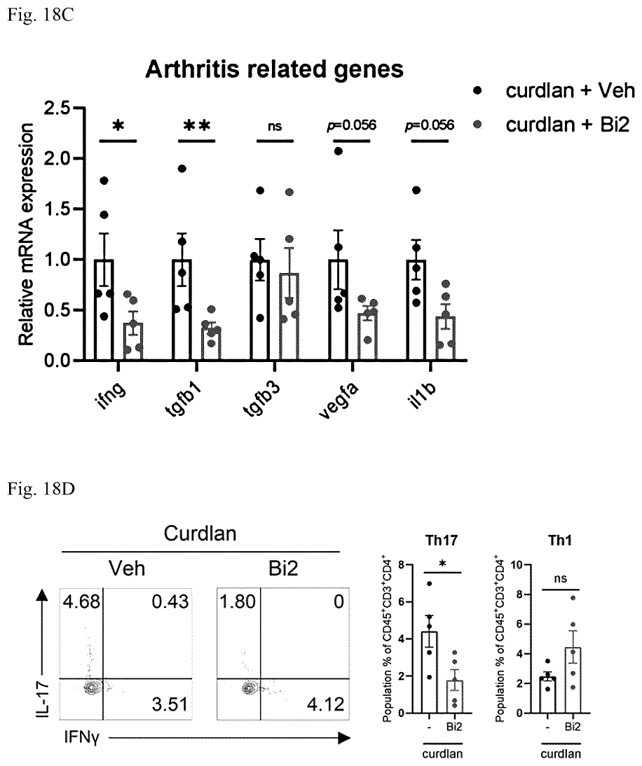

Next, the effect of SLC7A5-mediated BCAA influx on CD4⁺ T-cell response was investigated. SLC7A5 and BCAT1 expression increased to a similar extent in TCR-activated naive T-cell and memory CD4⁺ T-cell (FIG. 4A). Proliferation of these cells was comparable under various conditions resulting in changes in SLC7A5-mediated BCAA influx and metabolism. JPH203 completely blocked the proliferation of CD4+ naive and memory T-cells, but the depletion of BCAA in the culture medium results in partially reduced proliferation (FIG. 4B). On the other hand, no effect on proliferation was observed in TCR-stimulated CD4+ naive and memory T-cells pretreated with BCATc inhibitor 2 (Bi2), a BCAT1-specific inhibitor (FIG. 4B). The inhibitory potential of Bi2 was evaluated by quantifying the expression of alpha-ketoisocaproate (α-KIC), a metabolite of BCAT1, in HepG2 cells. HepG2 cells are a human liver cell line known for consistent and abundant BCAT1 expression (FIG. 3). The effects of SLC7A5 mediated influx and metabolic changes in BCAA on cytokine production were dependent on CD4⁺ T-cell subsets and cytokine profile. IL-2 production by CD4⁺ naive T-cells was not affected by JPH203 or Bi2 treatment or BCAA depletion, but IL-17A and IFN-γ production by CD4⁺ memory T-cells decreased significantly after JHP203 treatment (FIGS. 4C and 4D). In particular, BCAA depletion and Bi2 treatment preferentially inhibited IL-17A production (FIG. 4D), suggesting that intracellular BCAA and its metabolites are key to IL-17A production in CD4+ memory T-cells. To further determine which BCAAs are important for regulating proliferation and cytokine production, TCR-stimulated CD4⁺ memory T-cells were cultured in leucine, isoleucine, or valine-depleted media. Although each BCAA depletion had minimal effect on cell survival, inhibitory effects on proliferation of CD4⁺ memory T-cells and cytokine production were observed except for IFN-γ production in leucine-depleted media (FIGS. 5A to 5C). Thus, leucine depletion tends to affect IL-17A production. Given that leucine is involved in regulating the activity of mTORC1, a major signaling pathway for IL-17A expression, we decided to focus on the role of SLC7A5-mediated leucine influx by BCAT and its metabolites in the Th17 response. One of the interesting observations in this study is that the uptake and degradation of BCAAs, especially leucine, preferentially affect IL-17 production in human CD4⁺ memory T-cells (FIGS. 4C to 4E and 5C). Leucine depletion in culture medium significantly reduced IL-17A production by CD4⁺ memory T-cells upon TCR stimulation but not IFN-γ, and this was compared to conventional complete RPMI-1640 medium supplemented with 50 mg/ml leucine (FIG. 4E). Blocking or silencing BCAT1 inhibited IL-17A production even in media containing sufficient leucine (FIGS. 4E to 4G). Phosphorylation of S6 kinase (S6K), an important downstream substrate of mTORC1 for IL-17 production, was dependent on the presence of leucine in the medium (FIG. 5D). These data results indicate that the influx of BCAA through SLC7A5 and cytoplasmic metabolism are important in regulating IL-17A production in human CD4⁺ memory T-cells.

BCAT undergoes an amine group transfer reaction with leucine to form α-KIC, and at the same time, glutamate is produced from α-ketoglutarate (α-KG). In mitochondria, α-KIC is oxidized to isovaleryl-CoA mainly by the BCKD complex, resulting in the formation of final metabolites including HMG-CoA, acetoacetate, and acetyl-CoA46. Alternative pathways of leucine degradation in the cytoplasm have been reported, wherein α-KIC is converted to β-hydroxy s-methyl butyric acid (HMB) by KIC dioxygenase (also known as 4-hydroxyphenylpyruvate dioxygenase (HPD) or 4-hydroxyphenylpyruvate dioxygenase-like protein (HPDL) with dioxygenase activity similar to HPD46. Silencing HPD with shRNA showed a similar effect on BCAT1 inhibition, as the effect of HMB, a cytoplasmic leucine metabolite, on the regulation of IL-17A production was demonstrated. Moreover, the decrease in IL-17A production identified upon HPDL knockdown highlights that HPDL, which has an enzymatic activity similar to HPD, is involved in the regulation of cytoplasmic leucine metabolism.

### Example 4

### Regulation of Th17 Response of BCAT1-Mediated Leucine Metabolite HMB

HMB is a key metabolite of cytoplasmic leucine metabolism, and plasma levels depend on leucine-rich diet intake. HMB was used as a dietary replacement for leucine because HMB may improve muscle protein synthesis through mTORC1 activation and attenuate muscle protein degradation to promote muscle anabolism than leucine. It is noteworthy that HMB-induced mTORC1 activation is independent of the leucine sensing pathway mediated by Cestrin2.

Reanalysis of public RNA-Seq data showed that expression of BCAA metabolism related genes, particularly leucine, was induced in human TCR-activated CD4⁺ memory T-cells (FIG. 6A). qPCR confirmed that TCR stimulation increases expression of HPDL and BCKDK genes. The 4-hydroxyphenylpyruvate dioxygenase-like protein encoded by the HPDL gene has a dioxygenase activity similar to that of HPD and is involved in the production of the leucine metabolite β-hydroxy-β-methyl butyrate (HMB) (FIGS. 6A and 6B). In the cytoplasmic leucine metabolism pathway, HMB is known to be further metabolized to HMG-CoA and participate in the neosynthetic synthesis of cholesterol and acetyl-CoA (FIG. 6C). It regulates IL-17A production in CD4⁺ T-cells. Thus, the effect of this duleucine end product on IL-17 production in CD4⁺ memory T-cells was investigated. There was a slight increase in cholesterol after blocking BCAT1 with Bi2 treatment, and this increase was inhibited with HMB supplementation (FIG. 6D). Treatment with statins, mevalonate synthesis inhibitors, as previously reported, significantly reduced cholesterol and IL-17A production. In particular, Bi2 further reduced IL-17A production independent of cholesterol levels in CD4⁺ memory T-cells treated with statins (FIGS. 6D and 6E). It has been demonstrated that acetyl-CoA enhances the Th17 response through epigenetic reprogramming. We found that exogenous acetate dose-dependently reduced IL-17A production in CD4⁺ memory T-cells treated with Bi2, which was expected to increase intracellular acetyl-CoA levels (FIG. 6F). On the other hand, treatment with exogenous acetate upregulated IFN-γ production in CD4⁺ memory T-cells stimulated with Bi2-treated TCRs (FIG. 7A). HMB supplementation partially reversed the decrease in IL-17A production due to Bi2 in CD4+ memory T-cells in a dose-dependent manner (FIG. 6G). However, IFN-γ production was not affected by BCAT1 blockade or HMB supplementation (FIG. 7B). The effect of HMB was demonstrated by silencing HPD and HPDL with shRNA, which significantly inhibited IL-17A production (FIGS. 3H and 31). These results data indicate that HMB, a cytoplasmic leucine metabolite, contributes to increased IL-17A production in human CD4⁺ T-cells upon TCR stimulation.

In this study, Th17 cell production in CD4⁺ memory T-cells was significantly reduced by BCAT1 inhibition with Bi2, but this reduction was resolved by exogenous HMB treatment, suggesting that HMB plays an important role in the regulation of the Th17 response (FIG. 6G). In the cytoplasmic leucine metabolism pathway, HMB may be further metabolized to HMG-CoA, which participates in de novo cholesterol synthesis. Considering that cholesterol intake and synthesis are important for Th17 differentiation and response, the decreased IL-17 production observed in response to Bi2 may be due to decreased cholesterol synthesis. As expected, statins, a pharmacological inhibitor of HMG-CoA reductase, a rate-limiting enzyme of de novo cholesterol synthesis, reduced intracellular cholesterol levels and decreased IL-17A production (FIGS. 6D and 6E). However, Bi2 treatment significantly increased intracellular cholesterol levels in TCR-activated CD4⁺ memory T-cells, while exogenous HMB decreased cholesterol levels in these cells (FIG. 6D). These data suggest that BCAT1-mediated leucine metabolism does not affect intracellular cholesterol synthesis and that IL-17A regulation by cytoplasmic leucine metabolism is independent of de novo cholesterol synthesis in human CD4⁺ memory T-cells.

Cytoplasmic leucine metabolism also supports the synthesis of de novo acetyl-CoA, which is reversibly converted in HMG-CoA. Acetyl-CoA is known to support supplying fatty acids to proliferating T-cells, but enhances the Th17 response through epigenetic reprogramming. However, this study showed that BCAT1 inhibition did not affect CD4⁺ T-cell proliferation (FIG. 4B), and acetyl-CoA supplementation did not restore IL-17 production after BCAT1 inhibition (FIG. 6F). These data support a direct contribution of HMB to the regulation of IL-17 production. The concentration of HMB (400 μM) used in this study is the plasma level achievable after oral administration of HMB to healthy adults. This HMB plasma level reaches a peak within 60-120 minutes and the plasma half-life is about 2.5 hours. Given that treatment with exogenous HMB for 1 hour counteracted the inhibition of mTORC1 activity by Bi2 (FIG. 10G), there is a possibility that HMB supplementation affects human T-cell responses. One study showed that T-cells were hyperactivated in BCAT1^{-/-} mice, resulting in increased glycolytic metabolism and increased ATP synthesis capacity. Furthermore, these T-cells are likely to have reduced leucine amine conversion and increased intracellular leucine concentration, resulting in enhanced activity of the mTORC1/4EBP1 signaling pathway. As a result, BCAT1 is presumed to be a component of the negative feedback loop that regulates leucine availability for mTORC1 regulation in T-cells. On the other hand, this study revealed that inhibiting BCAT1 with a chemical does not induce overactivation of human CD4⁺ T-cells upon TCR stimulation (FIGS. 4B to 4F). This study showed that leucine was supplemented at a low concentration of 10 mg/L to restore leucine dependent Th17 production in CD4⁺ memory T-cells, reflecting leucine physiological levels in human plasma (FIG. 4C). The concentration of intracellular leucine required to directly activate mTORC1 may be limited, and the involvement of mTORC1 activity in Th17 production in human T-cells may involve both intracellular leucine and metabolites through the HIF1α dependent pathway. Recent studies have shown the inhibitory effect of pharmacological BCAT1 inhibition with new drugs on effector function of human CD8+ T-cells in vitro. In particular, this inhibition leads to a metabolic shift towards enhanced OXPHOS, highlighting the importance of BCAT1 in effector function, including differentiation of CD8+ T-cells and cytokine production.

### Example 5

scRNA-Seq Analysis

The scRNA-seq analysis revealed a unique signaling pathway involved in BCAT1-mediated regulation of IL-17A production. To explore the molecular mechanisms underlying BCAT1-mediated regulation of IL-17A production, scRNA-seq analysis was performed in human CD4⁺ memory T-cells with and without Bi2 at 72 hours after TCR stimulation. Unsupervised clustering and t-distributed stochastic neighbor (t-SNE) plot analysis allowed us to determine cluster identity based on the expression of established markers (FIGS. 8A and 9A) and successfully identify key CD4⁺ memory T-cell subsets, which were further grouped according to the expression of human T-cell activation signatures (FIGS. 8B and 8C). It was observed that a subset of Th17 cells showed relatively high expression of leucine metabolism related genes including BCAT1, SLC7A5 and HPDL compared to Th1 or Treg cells (FIG. 9B). Our data show that the activated Th17 cell population consisting of 8, 13, and 15 clusters decreased after inhibiting cytoplasmic leucine metabolism with Bi2 treatment (FIG. 8C). Pathway enrichment analysis of differentially expressed genes (DEGs) was performed to further analyze the biological significance of Bi2-mediated transcriptional changes in Th17 subsets. We detected a decrease in T-cell activation, cytokine signaling, and cytokine production pathways following Bi2 treatment in the activated Th17 subset (FIG. 8D). This pathway was primarily driven by downregulation of HIF1A, STAT3, and BCL2 (FIGS. 8E, 9C, and 5D). The expression pattern of HIF1A was projected onto t-SNE plots of activated Th17 cells (clusters 8, 13 and 15). Feature plots and violin plots showing that HIF1A expression is down-regulated in activated Th17 cells after Bi2 treatment are shown (FIG. 8F). Next, gene set enrichment analysis (GSEA) was performed on differentially expressed genes (DEGs) in Bi2-treated T-cells to gain mechanistic insight into the genes identified by scRNA-Seq analysis. There were 15 enriched pathways obtained from 23,457 DEGs identified in the activated Th17 cluster of TCR-activated CD4⁺ memory T-cells (p<0.1 and FDR<0.25) (FIG. 8G). Immune response related GSEA pathways such as complement, cholesterol homeostasis, PI3K-AKT-mTOR signaling, TGF-β signaling, IL-2/STAT5 signaling, and IFN-γ responses were included. Considering that leucine and its metabolites play a regulatory role in mTORC1 signaling activity in immune cells, we generated GSEA enrichment plots and heatmaps of DEGs involved in the downregulated PI3K-AKT-mTOR signaling pathway in Bi2-treated cells (FIG. 8H). A negative normalized enrichment score (NES) indicates enrichment in activated Th17 cells.

This scRNA-seq analysis supported the result that inhibition of cytoplasmic leucine metabolism with Bi2 had a major effect on the activated Th17 cluster and the mTORC1-HIF1α axis (FIG. 8). HIF1α is an important signaling molecule in the immune response, and its activity is coordinately regulated by the balance of transcription, translation and degradation. Immune cell activation, such as TCR or TLR binding, increases HIF1α expression via mTORC1 signaling leading to metabolic rewiring, which is important for effector function of immune cells. It has been reported that HIF1α enhances the direct transcriptional activation of RORγt and recruits the p300 complex with RORγt to the IL-17A promoter to regulate the Th17 signature gene. In the EAE mouse model, HIF1α^{-/-} CD4⁺ T-cells lack IL-17A production but not IFN-γ production in the spleen or lymph nodes, indicating resistance to EAE32. In this study, we confirmed that BCAT1 inhibition attenuates TCR-induced HIF1α expression in CD4+ memory T-cells and that exogenous HMB abrogates this attenuation at the transcriptional level by increasing the activity of the mTORC1/S6K axis (FIGS. 10A, 10B and 10E), resulting in increased expression of HIF1α target genes and IL-17A production (FIGS. 10C and 10D). The fact that a VHL-HIF-α interaction inhibitor named VH298 restores the IL-17A reduction seen after BCAT1 inhibition suggests that HIF1α plays an important role in reducing IL-17 production (FIG. 10D).

### Example 6

HIF-1α Regulation of HMB Via mTORC1 Activation in Human CD4+ T-Cells

TCR stimulation induces HIF-1α expression in human CD4+ memory T-cells at 24 hours post stimulation (FIG. 10A). This induction was significantly inhibited by blocking cytoplasmic leucine metabolism with Bi2 or silencing BCAT1, HPD, and HPDL with shRNA (FIGS. 10A and 10B). HMB supplementation reversed Bi2-mediated HIF-1α reduction (FIG. 10C), and this effect gradually decreased by 8 hours after HMB supplementation. This finding was demonstrated by qPCR analysis of the expression of Hif-1α target genes (FIG. 10D). Expression of SCL2A1, LDHA and PGK1 mRNA was reduced by Bi2, and this reduction was eliminated by HMB supplementation. Next, we investigated whether changes in HIF-1α expression mediated by Bi2 or HMB contribute to IL-17A production in CD4+ memory T-cells. The decrease in IL-17 production induced by Bi2 was fully restored by VH298, a VHL inhibitor that induces accumulation of HIF-1α29. However, treatment with Bi2, HMB or VH298 did not affect IFN-γ production (FIG. 10E). These results suggest that HMB increases the expression of HIF-1α in human CD4⁺ memory T-cells, thereby increasing IL-17A production.

HIF-1α is dynamically regulated through the balance of transcription, translation and degradation. To better understand the mechanisms underlying BCAT1-mediated HIF-1α regulation, several inhibitors were used to block the degradation pathway of HIF-1α, such as CoCl₂, an HIF-1α hydroxylation inhibitor, VH298, an E3 ubiquitin ligase pVHL inhibitor, and MG132, a proteasome inhibitor (FIGS. 11A and 11B). As expected, each inhibitor led to the accumulation of HIF-1α in TCR-stimulated CD4⁺ memory T-cells. However, none of these inhibitors affected the reduced expression of HIF-1α caused by Bi2. Thus, we tested whether HMB-mediated Hif-1α regulation was due to translational control. After 24 h pretreatment with Bi2 to deplete endogenous HMB, CD4⁺ memory T-cells were treated with cycloheximide (CHX), a protein synthesis inhibitor, in the absence or presence of HMB and analyzed for the rate of HIF-1α proteolysis (FIG. 11C). Consistent with the results shown in FIG. 10C, HMB supplementation increased expression of HIF-1α but did not affect HIF-1α degradation rate, suggesting that an increase in HIF-1α mediated by HMB is dependent on transcriptional regulation. qPCR revealed that HIF-1α mRNA expression was reduced by Bi2 treatment, and this reduction was rapidly abolished by HMB supplementation in human CD4⁺ memory T-cells (FIG. 10F). TCR-induced mTORC1 activity in human CD4⁺ T-cells results in HIF-1α protein accumulation through increased HIF-1α mRNA expression. Thus, the activity of p70-S6K, a sub-molecule of mTORC1, was monitored after Bi2 treatment. Immunoblot analysis showed that the inhibitory effect of Bi2 treatment on S6K phosphorylation in CD4⁺ memory T-cells was observed about 1-2 hours, relatively early after stimulation (FIGS. 11D and 10G). This inhibition of mTORC1 activity by Bi2 was time dependently counteracted by HMB supplementation (FIG. 10H). These data results demonstrated that HMB, a BCAT1-mediated leucine metabolite, increased mTORC1-dependent HIF-1α expression in CD4⁺ memory T-cells.

HIF-1α is also an important transcription factor in the differentiation program of naive CD4+ T-cells. Due to TCR-induced SLC7A5 and BCAT1 mRNA expression upregulation in human naive CD4+ T-cells (FIG. 4A), purified naive CD4⁺ T-cells differentiated into Th1 or Th17 cells under in vitro polarization conditions. Like CD4⁺ memory T-cells, BCAT1 inhibition reduced the activity of mTORC1 and the expression of HIF-1α (FIGS. 11E and 11F). Treatment with Bi2 also had no significant effect on the viability of activated human CD4+ naive T-cells (FIG. 11G). The results of the present study indicate upregulation of BCAT1 mRNA expression, particularly in Th17-polarized cells, indicating potentiation of cytoplasmic leucine metabolic activity (FIG. 11H). As a result, Bi2 treatment under Th17-polarized conditions significantly reduced mRNA levels of RORC and IL17A and consequently decreased IL-17A secretion (FIGS. 11I and 11J). Furthermore, IFNG and TBX21 mRNA expression and IFN-γ secretion by polarized Th1 cells in vitro were also reduced by BCAT1 inhibition (FIGS. 11K and 11L). Taken together, these data results show that HMB, a cytoplasmic leucine metabolite, upregulates the expression of HIF-1α through an increase in mTORC1 activity, which is closely linked to the regulation of CD4⁺ T-cell differentiation and function.

### Example 7

### Improving EAE Induction Through BCAT1 Inhibition

Consistent with the results observed in human CD4⁺ T-cells, TCR-stimulated T-cells increased mRNA expression of slc7A5 and bcat1 and protein expression of BCAT1 in mice (FIGS. 13A and 13B). Bi2 treatment preferentially inhibited the differentiation of Th17 cells but failed to inhibit IFN-γ production under each type of Th cell polarization condition (FIGS. 13C and 13D). To investigate the systemic in vivo effect of BCAT1 inhibition, an experimental autoimmune encephalomyelitis (EAE) model widely used to study the etiological response of autoreactive Th17 cells was adopted. Bi2 was administered intraperitoneally to MOG immunized mice 4 hours prior to immunization, and treatment was repeated three times a week for 14 days (FIG. 12A). Compared to control mice, Bi2-treated mice had significantly lower clinical scores from day 12 post-EAE induction, and this reduction was maintained until sacrifice (FIG. 12A). Consistent with the change in clinical score and total body weight (FIGS. 12A and 13A), histological analysis of spinal cord tissue showed a decrease in nerve damage (FIG. 12B) and cell infiltration (FIG. 12C) in Bi2-treated mice. The absolute number of spinal cord mononuclear cells (SCMC) and the proportion of CD4⁺ T-cells in SCMC in EAE mice compared to control mice were significantly lower after Bi2 treatment (FIGS. 12C and 12D, FIGS. 13B and 13C). More importantly, Bi2 treatment preferentially reduced the proportion and absolute number of CD4+ T-cells producing IL-17A among spinal cord infiltrating and draining lymph node (LN) CD4⁺ T cells (FIGS. 12E and 12F). Although Bi2 treatment did not change the relative proportion of pathogenic Th17 cells that co-produce IFN-γ/IL-17A, the absolute number was significantly reduced (FIG. 12E). This suggests that BCAT1 inhibition improves EAE induction by modulating the Th17 response of CD4⁺ T-cells in draining LN and CNS. Consistent with the results in human CD4⁺ T-cells, TCR stimulation increased HIF-1α expression in mouse LN T-cells and Bi2 treatment reduced TCR stimulation increase in HIF-1α expressing (FIGS. 13E and 13F). Immunoblot analysis showed that SCMC isolated from Bi2-administered mice had decreased HIF-1α expression (FIG. 12G). Importantly, as a result of intracellular staining for HIF-1α, this decrease occurred preferentially in CD4+ T-cells in SCMC but not in CD11b+F4/80+ macrophages (FIGS. 12H and 13D). These data show that HIF-1α regulation by BCAT1 in CD4+ T-cells is involved in EAE induction through Th17 cell generation.

### Example 8

### IL-17A Production Attenuation of LβhL, a Leucine Analog

Since Bi2 treatment affects BCAA metabolism through BCAT1 inhibition in an EAE mouse model, we sought to identify the role of cytoplasmic leucine bioavailability in modulating the Th17 response in this model. To this end, instead of SLC7A5 inhibitors that inhibit the influx of several essential amino acids, including leucine, leucine analogs were utilized as competitive inhibitors. L-β-homoleucine (LβhL) is a leucine analog that is efficiently transported by SLC7A5 but has minimal impact on mTORC1 activity. In addition, LβhL is known to be sufficiently stable for in vivo administration. Integration of ³H labeled leucine showed that LβhL competitively inhibited leucine influx in TCR-stimulated CD4+ T-cells (FIG. 15A). Similar to inhibition of BCAT1 by Bi2 treatment, LβhL did not affect proliferation of human CD4+ T-cells (FIGS. 15B and 15C), but LβhL preferentially reduced IL-17A production by CD4+ memory T-cells but not IFN-γ production and IL-2 production by CD4+ naive T-cells in humans (FIG. 15D). As shown in FIG. 7E, the induction and severity of EAE was attenuated in LβhL-treated mice compared to control mice to levels comparable to Bi2-treated mice (FIG. 15E). This finding was also confirmed by histological analysis of spinal cord tissue that revealed nerve damage (FIG. 16) and cell infiltration (FIG. 15G) was significantly lower in the LβhL treatment group than in the control group. Flow cytometry analysis showed that LβhL treatment significantly reduced the proportion of CD4+ T-cells producing IL-17A in spinal cord infiltrates (FIG. 15H) and draining LN CD4+ T cells (FIG. 15F). More importantly, HIF-1α expression was decreased in CD4+ T-cells in SC of LβhL treated mice (FIG. 15I). This is similar to that observed in Bi2 treated mice. These data highlight the importance of activation induced leucine influx and BCAT1-mediated leucine metabolism in CD4+ T-cells for the Th17 response regulating EAE induction.

### Example 9

### Arthritis Induced Improvement Through BCAT1 Inhibition

Eight-week-old female SKG mice used for induction of autoimmune arthritis [e.g., Rheumatoid Arthritis (RA)] were purchased from CLEA Japan (Tokyo, Japan). All mice were bred and managed in a sterile facility at Seoul National University School of Medicine. All experiments were approved by Seoul National University Institutional Animal Experimentation Ethics Committee (IACUC) (approval number: SNU-230825-2-2). At 8 weeks of age, 3 mg/mice curdlan (Wako, Japan) in 300 μl sterile PBS was injected intraperitoneally (i.p.) into mice. Ankle thickness was measured using calipers (Manostat, New York, NY). Edema of the joint area was observed in blind cages by two independent observers and evaluated as follows. 0 point: no joint swelling; 0.1 point: single finger joint swelling; 0.5 point: minor swelling of wrist, ankle or tail base; 1.0 point: severe swelling of wrist, wrist or tail base. Scores for all joints of each mouse were summed. Mice were euthanized at the end of the experiment and immune cells were extracted from inguinal lymph nodes (inguinal LNs) and joint cells.

BCAT1 inhibition (Bi2) showed an effect of alleviating autoimmune arthritis induced in SKG mice. Specifically, the arthritis score was significantly lower in the Bi2-administered group (*p<0.05), ifng, tgfb1 expression was significantly reduced in the Bi2-treated group, and the ratio of Th17 cells in CD3⁺CD4⁺ T cells in inguinal lymph nodes (iLNs) was significantly reduced (*p<0.005). Bi2 may alleviate arthritis symptoms by lowering arthritis score, reducing inflammatory gene expression, and inhibiting etiologic Th17 cells in a curdlan induced arthritis model of SKG mice.

What is claimed is:

1. A method for preventing, improving or treating an inflammatory disease; or an autoimmune disease, comprising administering to a subject a composition comprising an effective amount of a BCAT1 inhibitor.

2. The method of claim 1, wherein the inflammatory disease or autoimmune disease is one or more selected from the group consisting of autoimmune encephalomyelitis, multiple sclerosis, rheumatoid arthritis, systemic lupus erythematosus, type 1 diabetes, inflammatory bowel disease, Crohn's disease, ulcerative colitis, asthma, and atopic dermatitis.

3. The method of claim 1, wherein the BCAT1 inhibitor comprises one or more selected from the group consisting of BCATc inhibitor 2 represented by Formula 1, gabapentin represented by Formula 2, and ERG240 compound represented by Formula 3:

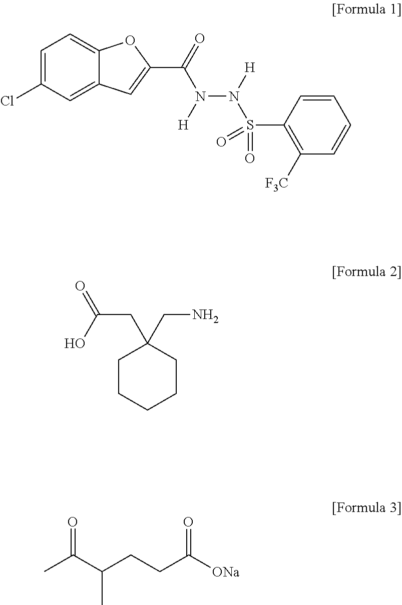

4. The method of claim 1, wherein a daily dosage of the BCAT1 inhibitor is from 0.1 to 100 mg/kg/day.

5. The method of claim 1, wherein the composition is a health functional food composition for preventing or improving an inflammatory disease; or an autoimmune disease.

6. The method of claim 1, wherein the composition is a pharmaceutical composition for preventing or treating an inflammatory disease; or an autoimmune disease.
